(19) United States
(12) Patent Application Publication
(10) Pub. No.: US 2014/0276995 A1
(43) Pub. Date: Sep. 18, 2014

(54) 4D Dynamically Contouring Mesh and Sutures

(71) Applicant: NOVO CONTOUR, INC., Edmonds, WA (US)

(72) Inventors: Michael P.H. Lau, Edmonds, WA (US); Leonard Pease, Bountiful, UT (US)

(21) Appl. No.: 13/831,159

(22) Filed: Mar. 14, 2013

(51) Int. Cl. A61F 2/00

(52) U.S. Cl. CPC A61F 2/0063
USPC 606/151

(57) ABSTRACT

A stressed timed-release multilayer composite, comprising a first stressed layer, and a second layer and third layer that hold the first layer under said stress. The second and third layers are configured to at least partially change to release at least a portion of the stress of the first layer in response to the second layer and/or the third layer being at least partially changed. Also disclosed is a stressed timed-release bilayer composite, comprising a first stressed layer and a second layer that holds the first layer under said stress forming a first physical curvature of the composite, wherein one or both of the first and/or second layers are configured to at least partially change and thereby form a second physical curvature. A stressed timed-release multilayer core-shell fiber is further disclosed.

### TECHNICAL FIELD

The present disclosure relates to synthetic or partially synthetic mesh for various uses, including, but not limited to, tissue support, tissue scaffolds, tissue replacements, bandages, sutures, and/or as elements in surgical meshes, sutures, and the like.

### BACKGROUND

Trends to translate surgical procedures employing large open incisions to minimally invasive surgery are firmly established. Smaller incisions translate into less tissue disruption along the path to the target tissue due to excision or subsequent repair, less traumatic surgeries for patients, and shorter and easier recoveries, with associated economic benefit.

To translate an open surgery to one that is minimally invasive, two requirements remain critical to make the transition possible, feasible, and successful. First, surgeons require access to and visualization (direct or indirect) of the target tissue to evaluate the tissues requiring treatment in the context of surrounding support tissue and organs. Second, surgeons require the ability to effectively perform the intended surgical procedures, including but not limited to excision, repair, or reinforcement, to achieve surgical objectives without damaging the underlying or surrounding tissue or organs. For those skilled in the art, these two requirements remain challenging to achieve skillfully and rapidly in many surgical procedures. While a surgeon can often see the target tissue in limited and confined spaces using ever smaller optical devices, the surgeon needs room to manipulate instruments to suture, staple, or plicate the tissue. Making room to accommodate these maneuvers creates more tissue trauma. Furthermore, although surgeons can see the target tissue using optical means, they cannot feel the target and the underlying tissue. Indeed, the tactile equivalent of open surgery, wherein the surgeon directly touches the patient's tissue (e.g., using fingers), remains elusive. However, many surgical maneuvers require this tactile feel to adjust tension, gauge the depth of penetration in placing sutures, and so forth, especially over critical structures such as nerves, blood vessels, bowels, or urinary tract. Very often, these vital structures remain in very close proximity to the target tissue to be treated.

Third, tissue curvature poses particular challenges for surgeons in confined spaces. Curvature, particularly where it varies with depth, may be difficult to visualize with many devices that present only a 2D view of the organs. Even where visualization is not limiting, surgical implements do not adequately mimic the natural or desired tissue curvature. For example, surgical mesh is often designed and delivered as flexible planar sheets that require suturing or plication to imperfectly approximate native tissue or organ curvature. Suturing and plication remain challenging to perform in tight spaces, leaving the repair imperfect and susceptible to failure.

Specific examples, among many possible examples, illustrating these challenges are illuminating. A first example concerns repair of cystocele from the field of pelvic surgery. Cystocele is one form of pelvic organ prolapse, for which currently nearly one-half million surgeries are performed in the United States each year. Cystocele, which commonly affects women, is caused by loss of bladder support from the anterior vaginal wall, allowing prolapse of the bladder into the vagina. Although persons less skilled in the art mistakenly assume the defect to be simply stretching and thinning of the anterior vaginal wall fascia and mucosa, a majority of the prolapse is due to the separation of the supporting tissue from the arcus tendineus fascia pelvis (ATFP), or the “white line” on the pubic bone that provides rigorous physical anchoring support to all anterior vaginal tissues. Any form of cystocele repair that simply plicates the loose tissue of the anterior vaginal wall (for example, via the trans-vaginal route), but without attachment back to the ATFP for solid anchoring, will frequently fail with rapid recurrence of the cystocele.

To perform paravaginal repair of cystocele (by attaching the supporting tissue back to the white line on the pubic bone to get solid anchoring), a surgeon can approach the repair either trans-vaginally or trans-abdominally. Trans-abdominal laparoscopic paravaginal repair remains technically challenging for many surgeons, requiring dissecting and suturing in tight spaces adjacent to extensive vasculature, with the bladder and urethra also nearby. Indeed, to properly complete a paravaginal repair, one has to dissect and clearly expose the white line to suture the supporting tissues to it. Yet, many blood vessels and the bladder remain in the way. Small errors rapidly become very bloody, presenting very real risk of damage or trauma to the bladder or urethra with extended recovery times. Open abdominal paravaginal repair may become necessary—a much more traumatic surgery with severe postoperative recovery periods. One may logically assume the trans-vaginal approach to be less invasive than the trans-abdominal approach. However, even the trans-vaginal approach remains similarly difficult due to the small spaces within the vagina, making exposure and surgical manipulation rather challenging. Very few gynecologists are trained to do trans-vaginal paravaginal repair.

To overcome those challenges in exposure and fixation, and to simulate traditional paravaginal repair, several commercial vaginal mesh kits have been developed that employ a thin trocar to deliver a mesh through an incision made through the vaginal mucosa to approach the white line on the pubic bone or the sacro-spinous ligament. Some mesh kits use a small anchor to attach the mesh to the ligament. Several problems in using these mesh kits have arisen. For example, reports indicate that the mesh caused tissue erosion, contraction, infection, pain, and dyspareunia. The deployment of the trocar and the anchor has been reported to cause damage to the bladder, urethra, blood vessels, and nerves in the operative areas, especially with the vessels and nerves behind the sacro-spinous ligament. An FDA warning relating to such mesh kits was issued, and litigation over the resulting complications remains widespread.

A second example concerns abdominoplasty to correct undesired belly protrusion, and derives from the field of plastic and cosmetic surgery. Abdominoplasty may be indicated due to excessive subcutaneous fat in the abdominal area or due to diastasis recti, the weakening of the muscular support of the abdominal wall muscle groups. In the latter case, simply performing liposuction and tightening the overlying skin will not offer desired aesthetic improvement because the abdominal contents still push out the abdominal wall. Open abdominoplasty, including the plication of the fascia and rectus muscle of the abdominal wall, corrects the protrusion problem secondary to diastasis recti. However, open surgery remains invasive and requires extensive postoperative recovery. Alternatively, endoscopic abdominoplasty provides a minimally invasive approach that induces less tissue trauma, offers shorter recovery, and reduces the extent of scarring, which may be more extensive by other means. Challenges with endoscopy include difficulty in exposing the large fascia plane overlying the weakened rectus muscle, difficulty in applying proper tension to plicate the fascia and muscle, especially with little room to adequately suture and adjust tension through endoscopic channels, and difficulty in confirming that the tension is neither too tight nor too loose because of the lack of tactile guidance available in open surgery.

A third example concerns repair of ptosis or drooping of the breast from the field of plastic and cosmetic surgery. Mastopexy remains a rather invasive procedure to lift the breast, leaving obvious scars after substantial recovery periods. Mastopexy remains one of the most problematic forms of aesthetic breast surgery, often with disputable results and impermanent resolution. Most ptosis of the breast is caused by weak fascial attachments that subsequently stretch the overlying skin as ptosis develops. Most minimally invasive mastopexies aim to create fewer obvious scars and correct the appearance of ptosis by simply tightening the skin. Without correcting the weakness in fascial attachment to provide reliable and robust support, gravity will readily restretch the skin with recurrent ptosis. It would be difficult to plicate or suspend the weakened fascial attachment of the breast due to the ill-defined nature of the facial tissue and the challenges in finding good anchors. Furthermore, excessive plication of the fascial tissue in the upper portion of the breast flattens the contour of that area which is not aesthetically pleasing.

For all of these needs, many of which are long standing, the present disclosure provides solutions in many forms, though one of ordinary skill in the art will understand and appreciate significant variations, combinations, and permutations thereon.

### SUMMARY

In various embodiments, the present application describes a composite material comprising two or more materials arranged in sheets, longitudinal elements, or mesh. At least one of the materials is stressed (i.e., tensioned or compressed) in one or more directions and held in tension or compression by at least one of the other materials. As supporting material is removed, the tension or compression causes the composite material to curve or bend out of plane in a temporally dynamic manner. In various embodiments, this composite material provides a specified temporally dynamic curvature to shape tissue.

In various embodiments, the present disclosure specifically provides solutions to a long-standing need for meshes that mimic and provide natural and desired curvature in the field of surgical treatment of pelvic organ prolapse, diastasis recti, urinary incontinence, and related maladies, among many other fields of use. Embodiments of the present disclosure further address the field of surgical bandages that, for example, conform and adopt the local curvature of a body system.

This foregoing summary is provided to introduce a selection of concepts in a simplified form that are further described below in the Detailed Description. It should be understood that this summary is not intended to identify key features of the claimed subject matter, nor is it intended to be used as an aid in determining the scope of the claimed subject matter.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 illustrates a gradual contouring of a three-layer system in views (a) to (f), in which a poly(vinyl alcohol) (PVA) layer initially holds a PDMS diamond mesh on a PLA frame flat. As the PVA layer dissolves, the PDMA-PLA composite gradually adopts increasing curvatures.

FIG. 2 illustrates contouring elements comprising two layers both (a) theoretically and (b) reduced to practice in a PDMS diamond mesh on a PLA frame.

FIG. 3 illustrates bending two materials into shapes, wherein (a) two uniformly adjoined materials with five equally spaced weak points allow the first tensioned layer to collapse the material into a hexagonal shape; and (b) two materials adjoined at five equally spaced points allow the first tensioned layer to collapse the material into a hexagonal shape. Cross sections represent either fibers or longitudinal elements.

FIG. 4 illustrates (a) contouring elements comprising three layers, at least in cross section; (b) releasing the stress stored in a central or middle layer causes the composite to bend with the bending direction governed by whether the central or middle layer is in tension or compression; (c) a trilayer system, at least in cross section, with divots or weak points on only one side folding into a square; and (d) a trilayer system, at least in cross section, with divots or weak points alternating sides folding into a zig-zag shape.

FIG. 5 illustrates folding 3D structures formed from sheets. Thin lines represent joints that bend the panels in a timed-release fashion.

FIG. 6 illustrates mesh designs that affect curvature, wherein (a) an anticlastic curvature is obtained from a regular hexagonal mesh, and (b) a synclastic curvature is obtained from a reentrant hexagonal mesh. Each element of the mesh may contain the composite disclosed herein.

FIG. 7 illustrates a picture of auxetic mesh formed in PDMS (a) before and (b) after tensioning.

FIG. 8 illustrates timed-release auxetic unit cells.

FIG. 9 illustrates a bilayer composite of length L in which the top layer thickness, strain, and modulus are given by h₁, ε₁, and E₁, respectively, and the bottom layer thickness, strain, and modulus are given by h₂, ε₂, and E₂, respectively.

FIG. 10 illustrates a schematic of a manufacturing process and in situ application process, showing (a) initially relaxed polymer strands within a fiber; (b) elongation and alignment of the polymer strands within a fiber under heat and with applied force that are then quenched or fixed into place by lowering the temperature below the glass transition temperature of the polymer; (c) attachment of the polymeric fibers to mesh and/or tissue; and (d) application of heat causing the fibers to recoil and decrease the length of the fibers so that the fibers apply a contractive force on the mesh and/or tissue, inducing a physical curvature in the same.

FIG. 11 illustrates a device to adjust mesh length during surgery comprising a positioning element, a heating element, and a length adjusting element, wherein (a) the length adjusting and heating elements may be on opposite sides of the device; (b) a 3D perspective of the length adjusting and heating elements; and (c) the length adjusting and heating elements may be on the same sides of the device opposed by a purely passive, insulating element.

FIG. 12 illustrates a synclastic curvature from (a) continuous triangular patterning; and (b) discrete triangular patterning.

FIG. 13 illustrates a curvature from (a) multiple layers contracted to different extents and (b) triangular contracting with decreasing contraction as the depth into the composite increases.

FIG. 14 illustrates a synclastic curvature induced by stressing one or more layers of a multilayer composite.

FIG. 15 illustrates a timed-release curvature on an expanding frame.

FIG. 16 illustrates an exemplary container that opens and shuts periodically by alternating the sign of the stress to adjust the curvature of a lid joint.

FIG. 17 illustrates open wound closure systems for (a) linear and (b) circular wounds viewed from the bottom and (c) from the side. The top of the wound closure system may comprise a membrane, while the bottom may comprise the microadhesive mesh. Microadhesive may be affixed to the mesh by suturing, taping, etc. (d) Initially flat bandages may achieve synclastic or anticlastic curvature by dissolving away an additional layer holding them initially flat.

FIG. 18 illustrates exemplary structures combining biomimetic and timed-released features including (a) hairpin and (b) FIGURE eight configurations of collagen-like fibers.

FIG. 19 illustrates exemplary mesh systems for repair of pelvic floor disorders such as cystocele. Similar combinations suffice for other pelvic floor disorders including rectocele.

FIG. 20 illustrates a mesh system for abdominoplasty to correct diastastis recti comprising biomimetic and timed-release mesh anchored to fascia with microadhesive.

FIG. 21 illustrates a mesh system for mastopexy comprising biomimetic and timed-release mesh anchored to fascia and clavicle bone with microadhesive.

FIG. 22 illustrates a curvature for a face lift that may be tuned following implantation by selectively applying energy.

### DETAILED DESCRIPTION

Traditional sutures are not dynamic in space or time. Once inserted within the body, they fix and hold specific tissues together at specific locations by design.

Unanticipated departure from these surgeon-specified locations may lead to unanticipated, adverse, and severe consequences. Although traditional sutures can degrade gradually if made out of biodegradable polymers, sutures do not dynamically change their three-dimensional (3D) configuration, curvature, or location. Similarly, traditional surgical mesh is not dynamic in space or time. It is designed to specifically anchor or support tissue in specific locations. Unanticipated departure from these surgeon-specified locations may lead to unanticipated, adverse, and severe consequences. Traditional surgical mesh can degrade gradually if made out of biodegradable polymers, but traditional mesh does not dynamically change its 3D configuration, curvature, or location.

However, in contrast to widely accepted teachings that sutures, mesh, bandages, and the like should not move following initial positioning by the surgeon or physician, these professionals may find specific instances wherein sutures, mesh, bandages, and the like that dynamically change in position over time or in time may be advantageous to their clinical practice as described below.

In various embodiments disclosed herein, the disclosed compositions of matter are dynamic both in time and position, hence the “four-dimensional” appellation “4D.” These compositions may comprise the entirety of structures, including for example, sutures, mesh, bandages, and the like, or specific elements of the same. Temporally dynamic positioning or curvature is accomplished by selective combination of materials that are tensioned, compressed, or neither, such that the composite adopts a first position or first physical curvature at a first time. One or more of the materials degrades, erodes, expands, swells, shrinks, delaminates, or changes its material properties relative to one or more other materials within the composite such that the composite adopts a second position or second physical curvature at a second time distinct from the first position or curvature.

Temporally dynamic positioning or curvature may also be accomplished by materials comprising one or more portions thereof that are tensioned, compressed, or neither, such that the overall material adopts a first position or physical curvature at an initial time. One or more of the portions of the materials degrades, expands, swells, shrinks, delaminates, or changes its material properties relative to another portion or portions such that the overall material adopts a second position or physical curvature at a second time distinct from the first position or curvature (see, e.g., FIG. 1). Hereafter the term “layer” refers to both materially distinct layers and portions of larger objects.

In various embodiments, the composites or portioned materials, also described herein as structures or compositions, change between positions or curvatures in a gradual manner. This is advantageous because damaged tissue supported by these structures also changes gradually but dynamically. In further embodiments, the structure changes abruptly between positions or curvatures. Abrupt changes may be achieved by selectively inducing localized delamination or by ratcheting through steady-states as multiple layers are removed sequentially. This may be advantageous if tissues need to be correctively rearranged, for example.

In various embodiments, the compositions or structures comprise two or more layers (see, e.g., FIG. 2). In some embodiments, one or more layers comprise polymers. In some embodiments, one or more layers comprise metals, ceramics, or nondegrading (on clinical time scales) polymer. In some embodiments, the materials are linearly elastic over the preferred strains and stresses. In some embodiments, a first layer 202 is stressed such that it extends under tensile forces or retracts under compressive forces. A second layer 204 is superimposed upon the first layer 202 in its stressed condition. As the applied initial tensile or compressive force is released, the first layer 202 partially (or negligibly) relaxes while the second layer 204 becomes partially (or negligibly) stressed with a sign opposite that of the first layer 202. If the first layer 202 is tensioned, then the second layer 204 becomes at least partially compressed. If the first layer 202 is compressed, then the second layer 205 is at least partially tensioned.

In some embodiments, a first layer 202 develops stress while a second layer 204 coupled to the first layer 202 retains the stress in the first layer. If the first layer is tensioned, then the second layer becomes at least partially compressed. If the first layer is compressed, then the second layer is at least partially tensioned. In both cases, because the two layers adopt opposing strains, a bending moment develops that causes curvature of the composition. As the dimensions, material properties, and/or stress-strain mismatch change, the curvature, position, and/or configuration of the composition also change.

In some embodiments, the layers are mostly planar such that a Cartesian coordinate system (with or without the Derjaguin approximation) would be natural for at least one configuration. In some embodiments, the layers would be considered flat and suitably represented by Cartesian in the absence of curvature. In some embodiments, the layers are initially flat such that the curvature is initially negligible. In some embodiments, the layers possess an initial curvature that is not negligible. In some embodiments, the curvature developed over time increases the initial curvature. In some embodiments, the developed curvature decreases the initial curvature. In specific embodiments, the developed curvature exceeds the magnitude of the initial curvature and is of opposite sign such that the composition curves out of plane in one direction and then curves out of plane in the other direction.

In various embodiments, the composition comprises individual strips or fibers (such as fibers having square or rectangular cross sections) that extend in a longitudinal direction such that the lateral or traverse dimensions remain less than the longitudinal direction. In various embodiments, the curvature develops in the longitudinal direction. In various embodiments, the curvature develops transverse to the longitudinal direction. In various embodiments, the composition comprises individual strips that extend in the longitudinal direction such that the lateral or traverse dimensions remain comparable to or greater than the longitudinal direction. In various embodiments, the curvature develops in the longitudinal direction. In various embodiments, the curvature develops transverse to the longitudinal direction.

In various embodiments, the approximately one-dimensional (at least in ratio) strips or fibers curve in two or three dimensions by controlling variations in the second layer. If the weak (i.e., thinned out and/or mechanically weaker/lower elastic moduli) points (or lines transverse to the object's primary axis) are arranged periodically on the layer on one side, then the bending may lead to the formation of a polygon (see FIG. 3). If the second layer comprises n-1 weak points, the strips or fibers form into an in-plane n-gon. If the n-1 weak points are equally spaced between the fiber or strip end points, a regular in-plane n-gon may form. For example, referring to FIG. 3a, including five divots 306 in the second layer 304 of a longitudinal strip allows the strip to bend into a hexagon 308 with the first tensioned layer 302 on the inside of the FIG. 1f initially in tension or the first tensioned layer on the outside if initially in compression.

In some embodiments, if the pre-stress or developed stress is locally enhanced at one location relative to a neighboring location, a bending moment also develops in the composition such that similar geometric figures, regular or irregular, may form. If the weak points vary in weakness or the degree of pre-stress varies between points, the timing and order of formation into 3D structures can be varied, controlled, and tuned. Similar variations in the first layer lie within the scope of the present disclosure.

In various embodiments, two or more strips or fibers 312 and 314 are connected (see FIG. 3b). In some embodiments, the strips or fibers are connected at discrete points 316. In some embodiments, the strips or fibers are connected continuously in specific regions. In some embodiments, the continuously connected regions are punctuated by regions without connectivity (i.e., delaminated). In some embodiments, the strips or fibers both deform within the same plane such that more intricate designs become feasible. For example, one fiber of a pair of fibers may form into a circle, while the second zig-zags, curves, or puckers so as to structure internal spaces within the circle 318 (see FIG. 3b). In some embodiments, the one or more strips or fibers deform within the plane while additional strips or fibers deform out of plane. In this manner, three-dimensional objects may be formed from essentially one-dimensional objects. One skilled in the art will recognize a multiplicity of variations based on these principles or similar to the aforesaid examples.

In various embodiments, the longitudinal strips 402 and 404 comprise additional layers 406. In some embodiments, one or more of these additional layers contains mechanical weak points, geometric weak points, or locally enhanced stress points such that local bending occurs. In some embodiments, if these points are arranged on the same side, the bending will lead to convex configurations 408 (see FIG. 4c). In some embodiments, if these points are arranged on alternating sides, the bending will lead to zig-zag configurations 410 (see FIG. 4c). In some embodiments, these points vary with the azimuthal direction relative to the longitudinal direction such that bending leads to spiral configurations. In this manner, three-dimensional objects may be formed from essentially one-dimensional objects. Continuous variation of these points allows for a wide variety of structures. One skilled in the art will recognize a multitude of variations based on these principles or similar to the aforesaid examples.

In various embodiments, the composition comprises layers that extend biaxially, like or similar to sheets. In various embodiments, the layers are continuous and uniform. In various embodiments, the layers are continuous but not uniform. In various embodiments, the layers are not continuous (i.e., discrete) but uniform. In various embodiments, the layers are neither continuous nor uniform. In various embodiments, the curvature develops preferentially in a first direction. In various embodiments, the curvature develops preferentially in a second direction. In various embodiments, the curvature developed in the first direction remains distinct in sign and/or magnitude from that developed in the second direction.

In some embodiments, the sheet-like layers bend sharply or gradually, curve, distend, or deform in and/or out of plane. For example, imposing or allowing a swath, divot, or “crease” characterized by locally attenuated thickness, locally enhanced or attenuated material properties, or regions of locally enhanced stress allows the sheet-like layers to bend toward the thinning if the first stressed layer is under tension or away from the thinned regions if the inner layer or if the first stressed layer is under compression. In some embodiments, the swath, divot, or crease spans the sheet. In some embodiments, the swath, divot, or crease does not span the sheet-like layers. In some embodiments, two or more creases, swaths or divots reside in the same second layer parallel to each other. In some embodiments, two or more creases, swaths or divots reside in the same second layer but at angles to each other. The composition bends along each crease. In some embodiments, the creases are linear or curvilinear. In some embodiments, the creases need not be linear.

In some embodiments, the sheet-like layers comprise additional layers. In some embodiments, the second and additional layers are strained to the same extent or not at all. In some embodiments, the second and additional layers are strained to different extents. In some embodiments, the second and additional layers reside on the same side of the first stressed layer. In some embodiments, the second and additional layers reside on opposing sides of the first stressed layer. In some embodiments, both the second and additional layers contain divots, swaths, or creases 502 on alternating sides. In this manner, the composition forms pleats or creases into fans or fan-like structures. In some embodiments, the composition gradually folds or curves into a wide variety of concave and convex configurations, shapes (e.g., boxes, icosahedra, cups, “Asian” fans, or a full array of 3D platonic and nonplatonic solids) or other containers 504, 506, and 508 (see FIG. 5). In some embodiments, the structures formed are geometrically regular. In some embodiments, the structures formed are geometrically irregular. For example, a cup may be formed from a degradable sheet with a thickness that decays radially on top of a biaxially tensioned sheet.

In various embodiments, the composition comprises mesh layers that extend biaxially, like or similar to sheets. In various embodiments, the mesh layers are continuous and uniform. In various embodiments, the mesh layers are continuous but not uniform. In various embodiments, the mesh layers are not continuous but uniform. In various embodiments, the mesh layers are neither continuous nor uniform. In various embodiments, the curvature develops preferentially in a first direction. In various embodiments, the curvature develops preferentially in a second direction. In various embodiments, the curvature developed in the first direction remains distinct in sign and/or magnitude from that developed in the second direction.

In some embodiments, the mesh is a diamond mesh. In some embodiments, the mesh is a square mesh. In some embodiments, the mesh is reentrant. In some embodiments, the openings in the mesh are square, diamond, rectangular, rectangular with rounded corners, circular, isosceles triangle, triangular, triangular with rounded corners, ovular, ellipsoidal, reentrant (with two materials of different thickness or three or more materials), reentrant square or cube or hexagon, curved or squashed reentrant cube, trichiral, fractal, laminate with multiple length scales, etc. (see FIG. 6). In some embodiments the curvature is synclastic. In some embodiments, the curvature is anticlastic.

In some embodiments, the mesh layers bend sharply or gradually, curve, distend or deform in and/or out of plane. For example, imposing or allowing a swath, divot, or “crease” characterized by locally attenuated thickness, locally enhanced or attenuated material properties, or region of locally enhanced stress allows the mesh layers to bend towards the thinning if the first stressed layer is under tension or away from the thinned region if the inner layer or first stressed layer is under compression. In some embodiments, the swath, divot, or crease spans the mesh. In some embodiments, the swath, divot, or crease does not span the mesh layers. In some embodiments, two or more creases, swaths or divots reside in the same second layer parallel to each other. In some embodiments, two or more creases, swaths or divots reside in the same second layer but at angles to each other. The composition bends along both creases. In some embodiments, the creases are linear or curvilinear. In some embodiments, the creases need not be linear.

In some embodiments, the mesh layers comprise additional layers. In some embodiments, the second and additional layers reside on the same side of the first stressed layer. In some embodiments, the second and additional layers reside on opposing sides of the first stressed layer. In some embodiments, the both the second and additional layers contain divots, swaths, or creases 502 on alternating sides. In this manner, the composition forms pleats or crease into fans or fan like structures. In some embodiments, the composition gradually folds or curves into a wide variety of concave and convex configurations, shapes (e.g. boxes, icosahedra, cups, “Asian” fans, or the full array of 3D platonic and nonplatonic solids) or other containers 504, 506, and 508 (see FIG. 5). In some embodiments, the structures formed are geometrically regular. In some embodiments, the structures formed are geometrically irregular. For example, a cup may be formed from a degradable sheet with a thickness that decays radially on top of a biaxially tensioned sheet.

In various embodiments, the mesh possesses auxetic characteristics. In some embodiments (see, e.g., FIGS. 6-8), the sheets or mesh 602 comprising reentrant structures 804 and 606 expand laterally when a uniaxial force is applied longitudinally in contrast to traditional materials 604 or mesh that contract laterally in response to a uniaxial force. In various embodiments, the lateral expansion is approximately negligible with auxetic or reentrant structures. In various embodiments, the lateral contraction is lessened with reentrant structures. All three conditions would be advantageous because they may present less biological disruption to adjacent tissues.

Stated differently, auxetic materials become thicker, not thinner, when stretched. In some embodiments, the disclosed auxetic or reentrant structures dynamically change their moduli and Poisson's ratios in time by including an additional removable layer 802 in contrast to traditional auxetic or reentrant structures that remain static or fixed.

In some embodiments, the lateral and longitudinal deformation, positioning and curvature are tuned separately by selectively tuning the local composition, material properties, dimensions, and initial and developed stresses. (e.g., within a unit cell.) In some embodiments, initially non-reentrant or non-auxetic structures become temporarily auxetic or reentrant by inducing curvature in the longitudinal arms. This is important because auxetic structures may allow for expansion when some segments or elements of the unit cell contract. Another advantage of including auxetic properties in mesh or other compositions is that the auxetic properties help contour the tissue in three dimensions. For example, materials having auxetic properties naturally adopt a synclastic curvature (see, e.g., Ugbolue, et al., Engineered Warp Knit Auxetic Fabrics, Journal of Textile Science & Engineering, 2 (2012)). According to still further embodiments, tissue coupled to dynamic auxetic mesh enhances its three-dimensional contouring effect (e.g., contouring the tissue around the jaw or breast).

In various embodiments, the composition comprises an additional layer. In some embodiments, one or more layers comprise polymers. In some embodiments, one or more layers comprise metals, ceramics, or nondegrading (on clinical time scales) polymer. This additional layer is in addition to the layers described elsewhere herein. In various embodiments, the additional layer provides an initial curvature. In some embodiments, the additional layer fixes the first two or more layers such that they lay flat. In some embodiments, the additional layer imposes a first curvature on the first two or more layers. In some embodiments, the additional layer is removed so that the curvature reverts to that of the first two layers alone. In some embodiments, the additional layer is then partially removed so that the curvature partially reverts to that of the first two or more layers alone. In some embodiments, the additional layer is removed by dissolving (see FIG. 1).

In some embodiments, the additional layer is removed by peeling it off. In at least one embodiment, the elastic modulus of the less tensioned or compressed layer(s) exceeds the elastic modulus of the tensioned or compressed layer(s) by approximately one order of magnitude. Differences of two to four orders of magnitude remain feasible. In some embodiments, the additional layer resides on the outside of the composition. In some embodiments, the additional layer lies on the inside except where the first two or more layers connect. In some embodiments, the additional layer comprises an interdispersed layer within the other layers such that when it is removed, the global structures relax. For example, a solid but dissolvable material within a foam. In some embodiments, the additional layer is connected by adhesion. In some embodiments, the additional layer is at least partially in direct contact with one or more of the other layers.

In various embodiments, the fibers or sheets comprise an internal layer not exposed to tissue or in vivo fluids, and an external layer that is exposed to tissues and bodily fluids. In specific embodiments, the external layer may comprise a glycocalyx or glycocalyx mimic. In specific embodiments, the external layer has sufficient thickness and material properties to tune the microstress environment to enhance desired cell growth and protein production. In specific embodiments, the external surface is composed of PEG or the like to minimize protein adhesion. In specific embodiments, the mechanical properties of the external layer range over 1−100 kPa so that collagen formation is minimized. In some embodiments, a sterilized gel having a lower modulus is included within or and around the composition.

In various embodiments, the external layer(s) contain pharmaceutical agents, biopharmaceutical agents, chemotractants, cell growth, or cell migration agents. For example, in deep wounds, the natural collagen matrix is disrupted such that fibroblasts cannot migrate deeply into the wound. Fibroblast migration may be encouraged by chemotractant release or by providing biochemical/biomechanical cues for migration. Similar cues govern the fibroblast to myofibroblast (i.e. mesenchymal) transition. Nerve growth can be channeled by similar means in conjunction with geometric or pathway cues. This disclosure incorporates the full array of molecules known to induce cell migration. In various embodiments, each layer of the composition may contain a distinct chemical composition such that each layer induces a distinct cellular response.

Various embodiments introduce a third or more layer(s). The third layer may comprise metals, ceramics, polymers, and the like. In various embodiments, second and third layers of the longitudinal strips, fibers, sheets, and/or mesh are symmetric. In various embodiments, second and third layers of the longitudinal strips, fibers, sheets, and/or mesh are not symmetric. In various embodiments, the second and third layers comprise frames about the first stressed layer. In various embodiments, the frames comprise complete sheets. In various embodiments, the frames comprise mesh. In various embodiments, two or more first stressed layers surround a second layer.

In various embodiments, the first stressed layer releases its stored stress energy in a timed-release manner. In some embodiments, the frame is at least partially removable. In some embodiments, the frame is configured to release at least a portion of the stored stresses in response to its removal. In various embodiments, the frame is removed by erosion or degradation. The erosion or degradation may be accomplished using mechanical, chemical, electrical, physical, or thermal processes or combinations thereof. For example, the erosion or degradation of the shell may include at least one of biodegradation, bioerosion, photooxidation, or photodegradation.

In some embodiments, tuning the composition's material properties and dimensions provides control over the other dimensions and the temperospatial profile of the composition. In various embodiments, the composition comprises medical products including, but not limited to, meshes, slings, bandages, sutures, tissue scaffolds, and the like. In various embodiments, the composition comprises elements thereof. In various embodiments, the second and third layers are comprised of sublayers or additional sublayers.

In various embodiments, multiple layers provide additional control or tunability to the positioning and curvature. In some embodiments, three or more layers of any composition within the scope and spirit of present disclosure allow for sequential timing of curvature and/or positioning. Thinner layers allow for more precise timing, while thicker layers of slower degrading material increase the duration over which the curvature or positioning develops. In some embodiments, multiple shell layers of modest thickness can be stacked to precisely control the degradation rate, curvature, and positioning in vivo. In some embodiments, different sections of mesh may curve in or out of plane relative to others. By straining different segments or sections of the mesh differently, some sections may shrink or expand at different rates or to different extents.

In various embodiments, the first layer comprises a cylindrical core or approximates a cylindrical core. In various embodiments, the first layer comprises a core that may be conveniently described in radial or cylindrical coordinates. In some embodiments, the second layer or shell partially or completely surrounds the first core layer. In some embodiments, the first layer or core is stressed in tension or compression. In some embodiments, the second layer or shell is stressed in tension or compression. In various embodiments, the core is comprised of one or more layers. In various embodiments, the shell is comprised of one or more layers. In some embodiments, one or more core or shell layers are comprised of metals, ceramic or nondegradable (at least on the times scale of the object's designed lifetime) polymer. In various embodiments, one or more of the core or shell layers are comprised of a removable polymer. In some embodiments, the removable polymer is biodegradable.

In some embodiments, the composition possesses an initial curvature along the core's longitudinal axis that is negligible. In some embodiments, the composition possesses an initial curvature along the core's longitudinal axis that is not negligible. In some embodiments, the developed curvature along the core's longitudinal axis increases the initial curvature. In some embodiments, the developed curvature decreases the initial curvature. In specific embodiments, the developed curvature exceeds the magnitude of the initial curvature and is of opposite sign such that the composition curves out of plane in one direction and then curves out of plane in the other direction.

In various embodiments, the approximately one-dimensional (at least in ratio) fibers curve in two or three dimensions by controlling variations in the second layer or shell (see FIG. 3). If the weak (i.e., thinned out or mechanically weaker/lower elastic moduli) points (or lines transverse to the object's primary axis) are arranged periodically on the shell on one side, then the bending leads to the formation of a polygon. If the second layer or shell comprises n-1 weak points, the fibers form into an in-plane n-gon. If the n-1 weak points are equally spaced between the fiber end points, a regular in-plane n-gon forms. For example, including five divots that completely or partially remove the second layer or shell on the fiber cores allows it to bend into a hexagon. (See FIG. 3).

In various embodiments two or more fibers are connected. In some embodiments, the fibers are connected at discrete points. In some embodiments, the fibers are connected continuously in specific regions. In some embodiments, the continuously connected regions are punctuated by regions without connectivity. In some embodiments, the fibers both deform within the same plane such that more intricate designs become feasible. For example, one fiber of a pair of fibers may form into an octagon, while the second zig-zags, curves, or puckers so as to structure internal spaces within the octagon.

In some embodiments, the one or more fibers deform within the plane while additional strips or fibers deform out of plane. In this manner, three-dimensional objects may be formed from essentially one-dimensional objects. In some embodiments, if these points are arranged on alternating sides, the bending will lead to zig-zag configurations. In some embodiments, these points vary with the azimuthal direction relative to the longitudinal direction such that bending leads to spiral configurations. In this manner, three-dimensional objects may be formed from essentially one-dimensional objects. Continuous variation of these points allows for a wide variety of structures. One of ordinary skill in the art will recognize a multitude of variations based on these principles or similar to the aforesaid examples.

In various embodiments, asymmetries in the shell thickness lead to curvature. In at least one embodiment, the shell may have a thickness that varies along the fiber length. For instance, a thinner or completely absent shell on one side of the core than the other leaves an imbalance in the mechanical forces. If the core is under tension, the core will contract where the shell is thinner, leading the whole structure to bend towards the thinner shell side. If the core is under compression, the core will expand where the shell is thinner, leading the whole structure to bend towards the thicker shell side. Similarly, the pre-stress or developed stress may be locally enhanced at one location relative to a neighboring location causing one or more bending moment(s) to develop in the composition such that similar geometric figures, regular or irregular, may form.

Each local region can have a different shell thickness such that bending can occur in multiple directions within or out of plane. In some embodiments, the fiber comprises a shell of uniform thickness, smoothly varying thickness, linearly increasing thickness, sinusoidally varying thickness, sigmoidally increasing thickness, exponentially increasing thickness, or mathematical summations/combinations thereof that leave the fiber with azimuthal and longitudinal asymmetries. In some embodiments, the core is not centered within the shell at one or more locations along the longitudinal axis.

In at least one embodiment, the fiber comprises a shell of a continuous gradient of material. In this manner, certain portions of the fiber may release their tension before other sections of the same fiber to apply the contraction/expansion and/or curvature more gradually or in a more targeted fashion. The (bio)degradation rate along with geometric, material, and mechanical factors control the timing and nature of the resulting curvature.

In various embodiments, the composition sustains weight. For example, if the anchor points are fixed and the composition is weight bearing, the composition will lift the weight. Alternatively, if the first stressed layer is in compression relative to the second, third, or additional layers, then upon removal of one or more of the these layers, the first stressed layer will expand. If the composition is anchored and the first stressed layer is somewhat rigid, the distance between the anchor points will increase. If the anchor points are fixed and the composition is weight bearing, the composition will lower the weight. In each case, removal of the outer layers releases the stored mechanical energy that can then act on the adjacent tissue. By tuning the fiber material properties, the release rate, and rate of removal, the mechanical effect of the composition can be controlled. The removal rate of the outer layer governs the rate of release of mechanical energy and the temperospatial profile of the first stressed layer, which in turn affects the position, configuration, and curvature of adjacent or included tissue.

Those of ordinary skill in the art will recognize and appreciate various combinations of these embodiments that lie within the scope and spirit of this disclosure. For example, the disclosed strips or fibers may be combined with sheets. Similarly, the disclosed mesh may be combined with sheets. In further embodiments, a mesh core may be combined with mesh frame. As a further embodiment, multiple fibers (cylindrical or layered) may be combined wherein the amount of pre-strain or pre-compression varies among the fibers within a core.

Various Ways to Tune the Curvature and Position

Stoney's formula for bilayer systems may provide a qualitative approximation of the curvature and deflection of embodiments disclosed herein. Zhang and Zhao (Journal of Applied Physics 99 (2006) 053513) derive Stoney's formula for the case of heteroepitaxial growth with lattice mismatch of one semiconductor layer on a substrate. Surprisingly and unexpectedly, though the physics remains very distinct and decidedly unrelated, the mathematical description is analogous. We consider a first layer 902 with prestrain εₚ (see FIG. 9) and a second adjoined layer 904. The average strains are then related by ε₁−ε₂=εₚ. Newton's third law then requires E₁ε₁h₁+E₂ε₂h₂=0. Solving for the strains finds ε₁=eₚE₂h₂/(E₁h₁+E₂h₂) and ε₂=eₚE₁h₁/(E₁h₁+E₂h₂). Because the strain in one layer possesses equal and opposite signs of the other layer, a bending moment develops that induces curvature of the composite. Zhang and Zhao show that the magnitude of the Stoney curvature then becomes κ=6E₁h₁εₚ/(E₂h₂²). This equation shows that the curvature decreases as the modulus and/or thickness of the tensioned layer decrease and as the modulus and/or thickness of the untensioned layer increase.

Increasing the imposed stress increases the curvature. The deflection may be approximated by assuming, as does Stoney, constant curvature (though constant curvature is not a limitation of the present disclosure). Then the sector length of a circle of radius R (=1/κ) equals the length of the composite, L, such that L=Rθ. In the limit of small angles, sin(θ=θ=Δz/L, where Δz represents the deflection of the mesh from the anchoring plane. Consequently, Δz=6E₁h₁eₚL²/E₂h₂². These formulas provide a qualitative indication of the key variables that govern the behavior of the composites herein, even though many of the embodiments disclosed herein are for trilayer, multilayer, or fibrous systems, although Stoney's formula requires numerous corrections to be quantitatively accurate for the disclosed system (e.g., for biaxial instead of uniaxial stress, inclusion of tissue and anchoring forces, etc.), corrections distinct from those available in the scientific literature.

In various embodiments, the curvature of the composite may be tuned or adjusted by affecting the pre-strain, εₚ. In some embodiments, the mesh prepared at ambient temperature when placed in the body will adjust the pre-strain due to thermal expansion. As each layer may possess distinct thermal coefficients of expansion, the strain mismatch represented by εₚ will be affected. If the increased temperature increases the pre-strain, the curvature increases. If the increased temperature decreases the pre-strains, the curvature decreases.

In various embodiments, the curvature may be tuned or adjusted by immersion in water. If the first layer has one level of hydrophilicity (or hydrophobicity) and the second or subsequent layers have a different level of hydrophilicity (or hydrophobicity), then the more hydrophilic components will swell or expand affecting the pre-strain and consequently the curvature of the composite in vivo. The rate and extent of curvature induction is controlled by the rate of hydration and the degree of hydrophilicity.

Similarly, electrostatic forces may play a key role. For example, polyelectrolyte hydrogels absorb substantially more water than neutral hydrogels. Therefore, composite mesh comprised of neutral and polyelectrolyte hydrogels will vastly change their curvature upon hydration. If the swelling increases the pre-strain, the curvature will increase. If the swelling decreases the pre-strains, the curvature will decrease. Similar but distinct arguments for other solvents follow similar analysis.

In some embodiments, the elastic moduli of the layers may be altered by release of a plasticizer. For example, triethylcitrate (TEC) and tributyl 2-acetylcitrate are an excellent plasticizers for poly(lactide) or poly(lactic acid) (PLA). These plasticizers are hydrophilic and water soluble such that they will gradually leach out of the PLA in aqueous environs in vivo. Leached TEC provides a protective effect to surrounding tissues, preventing fibrosis associated with PLA implantation. Generally, as the plasticizer departs, the elastic moduli increase. If the elastic modulus of a first layer increases relative to that of a second layer, the curvature of the composite will increase. If the elastic modulus of a first layer increases less than that of a second layer, the curvature of the composite will decrease.

Conversely, in some embodiments if one or more layers of the mesh are composed of polymer blends, the elastic modulus may gradually decrease over time. For example, if one or more layers comprises an interpenetrating polymer networks then the more hydrophilic of the two or more polymers will dissolve increasing the porosity of the network. Higher porosity materials tend to have lower elastic moduli, baring non-ideal thermodynamics or severe anisotropy. Similarly, polymers that contain discrete pockets or inclusions of hydrophilic materials including but not limited to small molecules, pharmaceutical agents, and more hydrophilic polymers, will also decrease in modulus as these materials dissolve or leach into the surrounding in vivo environment. The extent of porosity and modulus changes is directly affected by the processing of the mesh. If the elastic modulus of a first layer decreases more than that of a second layer, the curvature of the composite will decrease. If the elastic modulus of a first layer decreases less than that of a second layer, the curvature of the composite will increase.

In various embodiments, the dimensions of the composite and to some degree the elastic moduli may be affected or tuned by eroding or degrading one or more layers. To release the stored mechanical energy stored in one layer, other layers may be designed to degrade, biodegrade, bioerode, photooxidize, photodegrade, or otherwise oxidize or erode to release the stress in a controlled manner. For example, the erosion or degradation of the outer layer may include at least one of biodegradation, bioerosion, photooxidation, or photodegradation. Erosion or degradation may be further accomplished using mechanical, chemical, electrical, physical, or thermal processes or combinations thereof. Upon release of the tension, the composite contracts or expands and curves by a predetermined amount, in turn contracting, expanding, and curving the attached or adjacent tissue. Tunable erosion or biodegradation of polymer fibers is important to a well controlled mechanical energy release rate.

Biodegrading polymers come in two varieties: bulk-eroding polymers in which polymer erosion occurs simultaneously throughout their entire mass (i.e. both bulk and surface), and surface-eroding polymers in which only the exterior surface of the polymer undergoes degradation leaving the center intact. In at least one embodiment, the outer layer(s) is (are) composed of a bulk-eroding polymer. Here the rate of release of mechanical energy is governed, at least in part, by the local molecular weight of the polymer.

At early times, the molecular weight of the polymer is high, leading to substantial values of the elastic modulus. The elastic modulus of the shell should be at least of the same order of magnitude as that of the stressed layer. As the outer polymer bulk erodes, the polymer molecular weight decreases leading to successively lower values of the elastic modulus until the second, third, and/or additional layers are no longer able to restrain the expansion or contraction of the stressed layer and the mechanical energy stored therein is released.

Exemplary bulk-eroding polymers include polyesters (as defined by the presence of ester bonds) including but not limited to poly lactic acid (PLA), poly glycolic acid (PGA), poly(L-lactic acid) (PLLA), poly(D-lactic acid) (PDLA), poly(DL-lactic acid) (PLA or PDLLA), poly(caprolactone) or poly(ε-caprolactone) (PCL), and combinations thereof (e.g., poly(lactic-glycolic acid) (PLGA)), etc. In at least one embodiment, poly(lactic acid) is plasticized using diethylhexyl adipate, polymeric adipates (polyesters of adipic acid), polyethylene glycols of modest molecular weight, citrates, glucosemonoesters, partial fatty acid esters, poly(1,3-butanediol), acetyl glycerol monolaurate, dibutyl sebacate, poly(hydroxybutyrate), poly(vinylacetate), polysaccharides, polypropylene glycol, poly(ethylene glycol-ran-propylene glycol), dioctyl phthalate, tributyl citrate, adipic acid, thermoplastic starch, citrate esters, poly(ε-caprolactone), poly(butylene succinate), acetyl tri-n-butyl citrate, poly-(methyl methacrylate), poly(3-methyl-1,4-dioxan-2-one), diethyl bishydroxymethylmalonate, triethyl citrate, thermoplastic sago starch, oleic acid, glycerol, lactide monomer, lactic acid oligomers, triacetine, glycerol triacetate, monomethyl ethers of poly(ethylene glycol), dioplex, acetyl tri-ethyl citrate, and sorbitol. As indicated in the scientific literature, bulk-eroding polymers may also have a surface-eroding aspect as well, particularly where the polymer is at least partially hydrophobic.

In at least one embodiment, surface-eroding polymers may be preferred because bulk-eroding polymers may lose mechanical integrity rapidly and suddenly, leaving behind “chunks” of undegraded polymeric debris. In contrast, the biodegradation (i.e., bioerosion) rates of surface-eroding polymers may be more controllable and retain mechanical integrity until nearly all the polymer has eroded. For a surface-eroding polymer, the primary factor that governs the release of the energy in the stressed layer is the thickness of the outer degrading layer. As this layer thins, it is less able to resist release of the mechanical energy of the stressed layer(s). Eventually the second, third, and/or additional layers thin to the point where it can no longer resist the stressed layer(s), which then gradually expands or contracts to release its internal stress. As indicated in the scientific literature, surface-eroding polymers may also have a bulk-eroding aspect as well, particularly where the polymer is at least partially hydrophilic.

In at least one embodiment, two classes of well-studied polymers display surface erosion properties critical to maintaining mechanical integrity during a gradual, well-tuned degradation process: polyanhydrides and polymers formed by polycondensation reactions. The present application discloses members of both classes. Additional classes of surface-erodible polymers lie within the scope of this disclosure as newly discovered.

In at least one embodiment, the tensioned layer is comprised of poly(glycerol sebacate) (PGS) because it possesses elastin-like properties and can be easily and tunably stretched (i.e., pre-tensioned). PGS has been previously studied for a variety of applications (e.g., scaffolds for chondrocytes, myocytes, heart grafts, and retinal replacement). It has been found that NIH 3T3 fibroblasts grow nearly 50% faster on PGS than on polylactic-co-glycolic acid (PLGA), and further, a highly vascularized collagen forms around the implant in contrast to the fibrotic collagen that forms around PLGA. Additionally, PGS monomers have been approved for human use by the FDA because they are natural components of the lipid production cycle. Previous approval is advantageous because it decreases the time to clinic by accelerating the FDA 510k approval process. Millimeter thick PGS samples degrade completely in seven weeks in Sprauge-Dawley rats.

In at least one embodiment, the outer layer is comprised of polyanhydride, poly(1,3-Bis-(carboxyphenoxy)propane) (PCPP), because it can sustain organ weight similar to PLGA but has a linear degradation rate that is even slower than that of PGS. PCPP copolymers have also been approved by the FDA. In various embodiments, the PCPP resides on the external surface so that its degradation rate governs the first portion of the biodegradation process, the tension release timescale, and developed curvature, while the PGS controls the amount of composite contraction and the time to complete biodegradation. By controlling their respective thicknesses, the net degradation rate of the fiber will be highly tunable to achieve the targeted ½- to 24 month degradation window.

In another embodiment, the second, third, and/or additional layers are comprised of a polymer blend of two or more polymers so that the degradation time can be precisely tuned. For example, a mixture of PGS and PCPP or a mixture of PCPP with another polyanhydride, poly(1,3-Bis-(carboxyphenoxy)hexane) (PCPH), may be used to shorten or lengthen the degradation time relative to PCPP alone in a homopolymer melt. The mixture of polymers may be uniform and homogeneous or applied in separate coats to create lamina or gradients in the release rates so that the degradation time scale may be precisely controlled.

In at least one embodiment, the outer layer comprises surface eroding polymers including but not limited to poly(glycerol sebacate), poly(propane-1,2-diol-sebacate) (PPS), poly(butane-1,3-diol-sebacate) (PBS), A poly(butane-2,3-diol-sebacate) (PBS), A poly(pentane-2,4-diol-sebacate) (PPS), poly(1,3-Bis-(carboxyphenoxy)propane) (PCPP), polyanhydride, poly(1,3-Bis-(carboxyphenoxy)hexane) (PCPH), poly[1,6-bis(p-carboxyphenoxy)hexane], poly(sebacic acid)diacetoxy terminated, poly[1,4-bis(hydroxyethyl)terephthalate-alt-ethyloxyphosphate], poly[(1,6-bis(p-carboxyphenoxy)hexane)-co-sebacic acid], poly[1,4-bis(hydroxyethyl)terephthalate-alt-ethyloxyphosphate]-co-1,4-bis(hydroxyethyl)terephthalate-co-terephthalate), 1,6-Bis(p-carboxyphenoxy)hexane, other biodegradable polymers, and other polyester fibers formed by condensation and polyanhydrides.

In at least one embodiment, the first stressed layer comprises surface-eroding polymers including but not limited to poly(glycerol sebacate), poly(propane-1,2-diol-sebacate) (PPS), poly(butane-1,3-diol-sebacate) (PBS), poly(butane-2,3-diol-sebacate) (PBS), poly(pentane-2,4-diol-sebacate) (PPS), poly(1,3-bis-(carboxyphenoxy)propane) (PCPP), polyanhydride, poly(1,3-bis-(carboxyphenoxy)hexane) (PCPH), poly[1,6-bis(p-carboxyphenoxy)hexane], poly(sebacic acid)diacetoxy terminated, poly[1,4-bis(hydroxyethyl)terephthalate-alt-ethyloxyphosphate], poly[(1,6-bis(p-carboxyphenoxy)hexane)-co-sebacic acid], poly[1,4-bis(hydroxyethyl)terephthalate-alt-ethyloxyphosphate]-co-1,4-bis(hydroxyethyl)terephthalate-co-terephthalate), 1,6-bis(p-carboxyphenoxy)hexane, other biodegradable polymers, and other polyester fibers formed by condensation and polyanhydrides. In at least one embodiment, the first stressed layer is biodegradable, bioerodible, degradable, erodible, photooxidable, and/or photodegradable.

In at least one embodiment, the first stressed layer is comprised of non-biodegradable materials including but not limited to poly(dimethyl siloxane) (PDMS) (including, for example, silastic MDX4-4210 or MED-4210, inter alia), PDMS with silica (such as bionate 75A, bionate 2, bionate 75D and carbosil 80A, inter alia), polyisoprene, polyethylene oxide, natural rubber, latex, and polyurethane. In at least one embodiment, the first stressed layer(s) consists of polymer(s) having linear elastic stress-strain curves. In some embodiments, the second, third, and/or additional layers shells may be made from any biodegradable polymer including PEG, PCCP, PCHP, PLA, PLGA, PGA, PCL, etc., and plasticized materials of the same.

In various embodiments, dopants are used to tune the composition's material properties, dimensions and/or pre-stress. In various embodiments, the dopant leaches out, dissolves out, and/or diffuses out of the composition to change its configuration, position, and/or curvature. In various embodiments a porogen assists in tuning the composition's material properties, dimensions and/or pre-stress.

In various embodiments, energy sources are used to tune the composition's material properties, dimensions and/or pre-stress. These in turn tune the position and curvature of the composition. In various embodiments, energy is used to set or tune a curvature prior to implantation to tailor the implant to the patient. In various embodiments, energy is used to set, tune, or refine a curvature during implantation. In various embodiments, energy is used to set, tune, or refine a curvature following implantation.

In some embodiments, thermal energy or heat is used to tune the composition's material properties, dimensions, and/or pre-stress. In some embodiments, ultrasound is used to tune the composition's material properties, dimensions, and/or pre-stress. In some embodiments, radiofrequency sources are applied to tune the composition's material properties, dimensions, and/or pre-stress. In some embodiments, thermionic energy sources are used to tune the composition's material properties, dimensions, and/or pre-stress. In some embodiments, these energy sources increase the composition temperature (i.e., above the glass transition temperature) allowing it to relax internal stress (i.e., the pre-stress). In some embodiments, these energy sources increase the composition temperature, which in combination with applied stresses or tissue stresses leads to changes in the compositions dimensions or material properties. In some embodiments, increasing the temperature allows dopants, discrete inclusions of polymers or other materials, and/or plasticizers to diffuse more readily affecting the composition's prestress, dimensions, and material properties. These mechanisms provide the surgeon with complete control over the dimensions, positions, configurations, curvatures, and rates of change of dimensions, positions, configurations, and curvatures.

In various embodiments, the composition may be attached in one or more locations to prestressed fibers, shims, and/or similar elements (see FIG. 10). For example, shims or fibers 1002 sheared above their glass transition temperature and quickly quenched such that their molecules retain an extended conformation. As the fiber, shims and the like are again heated above their glass transition temperature, the extended conformations relax, decreasing the length of the fiber or shim. In this manner, the surgeon can selectively shrink or expand a mesh, suture or bandage 1004 after implantation. In some embodiments, the fibers, shims and the like are exposed at specific locations on one side of the composition such that the entire composition bends. In some embodiments, the fibers, shims and the like are exposed at one depth such that the composition bends preferentially in one direction. In some embodiments, the fibers, shims and the like are exposed at a second depth distinct from the first depth such that the composition bends preferentially in a second direction.

In some embodiments, the fibers are exposed using one energy modality such that the composition bends in a first direction and then exposed using a second energy modality so that the composition bends in a second direction or to a second extent. In some embodiments, the fibers are exposed using one energy level or frequency such that the composition bends in a first direction and then exposed using a second energy level or frequency so that the composition bends in a second direction or to a second extent. In some embodiments, the composition is doped such that it absorbs more energy of a first kind in a first layer or first portion. In some embodiments, the composition is doped in a second manner such that it absorbs more energy of a second kind in a second layer or second portion.

This ability to tune the fiber length is important because perfect placement is not always feasible leading to undesirable consequences. For example, if the mesh is under tightened it may not provide sufficient lift and/or support to resolve the underlying condition. If the mesh is over tightened, it can lead to undesirable surgical complications. The surgeon or physician may determine whether the initial position is acceptable. If not, the mesh requires adjustment in length. However, commercially available mesh cannot be adjusted once fixed in place. The surgeons may remove the mesh and try to replace it or release the hooks and reinsert them. This may lead to over tightening and/or additional tissue damage, causing additional surgical complications, and/or increasing the time to full recovery for the patient. Therefore, there is a clear need for the novel mesh, fibers, and sutures described herein that can be adjusted in length after surgical placement. The present disclosure represents a significant advance by giving surgeons an additional tool to improving surgical outcomes.

In some embodiments, the polymer fibers comprise two properties. First, they have a glass transition temperature between 40° C. and 55° C. The lower end of this range is governed by the need to have glass transition temperatures in excess of the upper range of normal body temperature or body temperature corresponding to a fever. A different lower temperature range may govern in veterinary or other bodies/environments. The upper range is governed by the need to minimize adjacent tissue damage. The upper temperature range may be lower if exposure for longer times is needed. The upper temperature range may expand if adjacent tissue is at least partially insulated or the exposure time is short.

In various embodiments, the preferred glass transition temperatures may be designed into the fibers by means of materials selection. In some embodiments, homopolymers that are suitably biocompatible for mesh or sutures may be selected without extensive modification. In some embodiments, the glass transition temperature of the fibers may be altered to be within the design range by inclusion of a suitable plasticizer. Plasticizers that do not persist for long periods of time are acceptable so long as the glass transition temperature remains within the desired range above for the duration required to modify the mesh to the desired length. This duration may be the time after initial surgical positioning or the time until surgical reintervention takes place days or weeks following initial surgical positioning.

In some embodiments, the glass transition temperature may be designed into the mesh by suitable selection of copolymers. The Gordon and Taylor equation copolymer equation may be used to determine the weight fractions of the two copolymers that will give the optimal polymer glass transition temperature. Here 1/Tgcopolymer=w1/Tg1+w2/Tg2, where wi is the weight fraction and Tgi represents the glass transition temperature of monomer i in the final polymer in the Kelvin scale. Tables 1 and 2 below provide examples of copolymer compositions that are generally considered to be suitable for implantation and that fall with the desired glass transition temperature range.

TABLE 1
Glass Transition Temperature of Common Biomedical Polymers

| Polymer | T_{g} (° C.) | Reference |
|---|---|---|
| PCL | −60 | http://en.wikipedia.org/wiki/Polycaprolactone |
| TMC | −18 ± 1 | Pego, et al., Polymer, 44 (2003) 6495-6504 |
| PGA | 37.5 ± 2.5 | http://en.wikipedia.org/wiki/Polyglycolide |
| PLLA | 62.5 ± 2.5 | http://en.wikipedia.org/wiki/Polylactic_acid |

TABLE 2
Exemplary Biomedical Copolymers with T_{g} = 40-55° C.

| | First Monomer | Second Monomer |
|---|---|---|
| Copolymer | Weight Fraction | Weight Fraction |
| PCL-PLLA | 0.04-0.12 | 0.88-0.96 |
| TMC-PLLA | 0.07-0.23 | 0.77-0.93 |
| PGA-PLLA | 0.28-0.89 | 0.11-0.72 |

In various embodiments, the fibers shrink when heated (see FIG. 10). In some embodiments, this may be achieved by aligning or elongating the polymer strands within the fibers during the fiber manufacturing process. In some embodiments, heating the fibers above the glass transition temperature (but below the melting temperature) of the polymer while applying a force will cause the fibers to align. Quenching the fibers below the glass transition temperature secures the strands in an aligned configuration. In some embodiments, the force may be applied normally at one or both fiber ends or alternatively, the force may be applied as a shear force near the surface. The former provides a uniform alignment throughout the fiber. The latter provides enhanced alignment at the fiber surface, particularly if there is a temperature gradient in the fiber with the outer portion at a higher temperature than an inner portion.

In some embodiments, the force should be applied quickly, where quickly is defined relative to thermal relaxation. The time scale for stress relaxation is given as the ratio of the polymer viscosity divided by the shear relaxation modulus (see, e.g., <http://www.files.chem.vt.edu/chem-dept/marand/Lecture20.pdf>). Alternatively, a number of stress relaxation times and correlations are available to assist in the design process (see Roland, et al., Determining Rouse relaxation times from the dynamic modulus of entangled polymers, Journal of Rheology, 48 (2004) 395). In some embodiments, the relaxation time scale should be of the same order of magnitude or greater than the process time scale, such that the Deborah number is of the same order of magnitude or greater than unity (see Polymer Processing Fundamentals By Tim A. Osswald). The process time scale may be the time during which the temperature exceeds the glass transition temperature, the time the shear stresses are applied, the increased length of the fiber divided by the velocity of extension, or the time after the shear stresses are applied but before the temperature falls below the glass transition temperature. The latter two process time scales are perhaps most relevant in the higher throughput manufacturing environments.

In some embodiments, alignment of the polymer within the fibers may be accomplished by several means available to those skilled in the art. For example, the fibers may be extruded above the glass transition temperature but with a suitably large Deborah number. In some embodiments, the fibers may be extruded normally (i.e., with modest Deborah number flows) and then post processed. In further exemplary processes, the fibers may be prepared by injection molding, blow molding, film blowing with cutting into narrow strips, thermo forming, and a variety of other processes known to those skilled in the art. The post processing to align the polymer strands within the fiber may be accomplished, for examples, in a tube furnace with the collection rate of the final spool in length collected per unit time exceeding that of the initial spool (see FIG. 10). In some embodiments, the fibers may be heated within a furnace, stretched, and then quenched.

In various embodiments, the fiber length may be reset by either stretching or shrinking the fibers under heat (see FIG. 10). In some embodiments, stretching the fibers under heat with an applied force is straightforward. In some embodiments, shrinking the fibers under the same heat source is nonintuitive but follows directly from the alignment of the fibers. When the aligned fibers are heated with minimal to negligible stress, the polymer strands relax from their extended, aligned state into entangled, random coil configurations. Above the glass transition temperature, the relaxation occurs because the polymer strands can increase their entropy at the expense of the enthalpic forces that hold the alignment below the glass transition temperature. At elevated temperatures the product of temperature and entropy exceed that of the enthalpy giving the relaxation an optimal free energy necessary for the spontaneous transition.

Various embodiments disclosed devices to adjust the composition's length (see FIG. 11). In some embodiments, the device at minimum sets the new desired length and applies thermal energy. In various embodiments, the device may also perform other functions, including, but not limited to, protecting patient tissue from thermal exposure, irrigating, etc. In some embodiments, e.g., as shown in FIG. 11, the device comprises a positioning element 1702. This element's purpose is to register the mesh or suture fibers 1706 between the two layers of the device and isolate the fibers from the patient's tissue. In some embodiments, this element is small to minimize the stresses applied to the tissue upon insertion. In some embodiments, this element reaches to the section of the mesh or suture that requires extension or contraction, and can preferentially be administered in a minimally invasive manner (e.g., laparoscopically).

In some embodiments, another element comprising the device adjusts the length (see FIG. 11). In some embodiments, this element comprises, first, a set of clamps, pins, or other means of attaching, fixing, or binding the mesh to the length adjustment element. Second, this element comprises a way of increasing or decreasing the device length. This may be achieve by a wedge to drive two parts of the element, a two part motorized stage, or other means of adjusting the length between the clamps.

In some embodiments, another element comprising the device is the heat application element 1704 (see FIG. 11). This element is important because it applies the thermal energy required to heat the polymer above its glass transition temperature. In some embodiments, this element may consist of one or more heating elements. In some embodiments, the heating element(s) may be controlled separate for uniform shrinkage or expansion or controlled independently at registered locations so that individual fibers of the mesh can be expanded or shrunk independently. Independent heating provides the surgeon complete control over the final three-dimensional arrangement of the fibers so that the physician can control the 3D curvature of the mesh or suture, cause compositions on the edge of the mesh to shrink more or less than the compositions in the center, or compositions on one side of the mesh to shrink more than those on the other side. Independent heating also provides an adjustable means of increasing or decreasing the length of fiber thermally exposed.

In various embodiments, the length adjustment element and the heating element may occur on opposite sides of the device (see FIG. 11). This arrangement is advantageous because they can be independently operated and are more straightforward to manufacture. In various embodiments, arrangement of both the length adjustment and heating elements on the same side of the device is also feasible (see FIG. 11). Then the opposing side may be purely passive. In some embodiments, an optional element of the mesh is a cooling element. This element may minimize thermal exposure to adjacent tissue. It may also be helpful in quenching the tissue mesh so that partial alignment can be maintained to prevent the mesh from completely shrinking. In at least one example, the cooling may be achieved by means of Peltier or thermoelectric cooling elements.

In various embodiments, the surgeon may select the heating and/or cooling profiles. In various embodiments, the method of operation of the device may follow at least two characteristic patterns. In a first characteristic pattern, the mesh is first sandwiched between two elements of the device. The fibers are clamped, fixed, or bound to the surface and the length is set to the new desired length by stretching. Heat is applied to raise the temperature of the mesh above the glass transition temperature. The heat may be applied before, after, or during stretching to the new desired length. The fibers achieve the new desired length. The time required for this to be achieved can be predetermined from time-temperature processing profiles. The fibers may be quickly quenched to body temperature so that the length does not continue to change.

A second characteristic pattern involves contracting the fiber length. Here, the mesh is first sandwiched between two elements of the device. The fibers are clamped, fixed, or bound to the surface and the length is set to the new desired length by shrinking so that the fibers become limp. Heat is applied to raise the temperature of the mesh above the glass transition temperature. The fibers achieve the new desired length. The time required for this to be achieved can be predetermined from time-temperature processing profiles. The fibers may be quickly quenched to body temperature so that the length does not continue to change.

According to further embodiments of the disclosure, energy can be applied to the contour zones to contract the composition (and thereby the adjacent target tissue) in three dimensions. For example, contour zones having a generally triangular shape or exposed regions arranged in a generally triangular pattern can achieve three-dimensional contouring of the composition and tissue. FIG. 12, for example, is a schematic diagram of a contour zone 1206 in accordance with embodiments of the disclosure. FIG. 12a illustrates the contour zone 1206 before applying energy to a single exposed region 1204 having a generally triangular shape 1202. FIG. 12a further illustrates the contour zone 1206 after applying energy to the triangular exposed region 1204. As illustrated in FIG. 12a, triangular shape of the contour zone 1206 results in preferential contraction in three dimensions.

FIG. 12b is a schematic diagram also illustrating a contour zone 1212 configured to contract the composition and target tissue in three dimensions. FIG. 12b illustrates the contour zone 1212 before applying energy to a plurality of exposed regions 1210 of tissue interspersed with unexposed regions configured in a generally triangular pattern 1208. FIG. 12b further illustrates the contour zone 1212 after applying the energy to the plurality of exposed regions 1210, such that the contour zone 1212 is also preferentially shaped in three dimensions. The contour zones 1206, 1212 including generally triangular patterns 1202, 1208 of exposed regions 1204, 1210 may vary in number or magnitude across the tissue. The illustrated configurations are useful embodiments because they can allow for control of the curvature of the tissue in three dimensions with a two-dimensional exposure pattern.

According to another embodiment of the disclosure, the depth of the energy exposure to the composition may also be adjusted to induce a three-dimensional curvature of the composition and target tissue. For example, the depth or intensity of exposure may differ in a single exposed region, or in one exposed region with reference to an adjacent exposed region. FIG. 13a is a schematic side cross-sectional view of a target tissue having an induced curvature due to different depths of energy application. FIG. 13a represents a target composition and tissue 1302 having energy applied at different depths, and FIG. 13a further represents the curved composition and target tissue 1302 after it has been preferentially contracted. In the illustrated embodiment, the energy is selectively applied to a first depth 1304 and to a second depth 1306 of the tissue 1302. The selective amounts of energy applied to varying depths of the tissue 1302 provide a net curvature of the tissue 1302, as illustrated in FIG. 13a.

FIG. 13b illustrates another embodiment of varying the depth of energy application to induce curvature in a composition and target tissue 1312. For example, FIG. 13b represents the energy exposure depths to the composition and target tissue 1312, and also represents the composition and target tissue 1312 after applying energy to contract the composition and/or tissue. In the illustrated embodiment, energy is applied to a plurality of exposed regions 1314 interspersed among non-exposed composition and/or tissue 1316. The energy penetrates the exposed regions 1314 such that the depth of the exposure has a generally triangular shape. Thus, exposing the composition and target tissue at selectively varying depths can also contour the composition and target tissue into the desired direction and shape including, for example, a convex curvature with reference from inside the tissue. One skilled in the art will appreciate, however, that the present disclosure is not limited by the exposure depths of the illustrated embodiments. For example, energy may be applied to three or more depths or to exposure depths having shapes other than triangular shapes.

FIG. 14 also illustrates the effect of varying energy exposure depth to contour the composition and tissue in three dimensions. More specifically, FIG. 14 comprises a top view of a contour zone 1402 and an isometric view of the contour zone 1402. The contour zone 1402 includes a first exposure region 1404 having energy applied to a first depth or intensity, and a second exposure region 1406 having energy applied to a second depth or intensity. The first and second exposure regions 1404, 1406 can accordingly have concentric elongated regions to achieve the preferred contraction in three dimensions.

Methods of Manufacture

This disclosure presents several exemplary ways and combinations thereof to construct, fabricate, and/or manufacture the disclosed compositions, without limiting the spirit and scope of the present disclosure. Those of ordinary skill in the art will recognize and appreciate additional way or methods of achieving the strips, fibers, sheets, mesh, etc., which remain within the scope and spirit of the present disclosure.

First, the first stressed layer may be placed in tension by purely mechanical means. For example, the first stressed layer may be stretched to a preferred length or to a preferred tension by external mechanical forces. Specifically, a first stressed layer may be clamped at its ends, and increasing the distance between the clamps applies a tension to the first stressed layer. The tension may be fixed in place by securing the first stressed layer with a second, third, or additional layer. The ends may be specifically annealed or affixed by a variety of means (e.g. tying, clamping, crimping, etc.) to the second, third, or additional layer to prevent delamination. Although shear forces between the first stressed layer and adjoining layers will cause or allow both to contract and/or curve, tension remaining in the core remains available to act on adjacent tissue or impose curvature on adjacent tissue after removal of the second, third, or adjoining layers,

Second, the first stressed layer may be placed in compression by means of swelling it. For example, the first stressed layer may be comprised of a dry-formed hydrogel. A non-swelling or minimally swelling second, third, or additional layer(s) may be applied to the dry hydrogel. Upon implantation in vivo or exposure to hydrating solutions such as water, the first stressed layer will swell, at least partially, building up compression within the first stressed layer as the second, third, or additional layer(s) resists the expansion due to the swelling. Removal of the second, third, or additional layers will release the compression allowing the first stressed layer to further swell and expand to oppose tissue contraction, extend the length of adjacent tissue, or impose curvature on the tissue. In this example, a range of hydrogel compositions are viable from simple uncharged hydrogels to polyelectrolyte hydrogels, inter alia.

In a further example of the same, the first stressed layer comprises a series of hydrogel rods having a central string or strand connecting the hydrogel rods. In various embodiments, the first stressed layer also comprises relatively stiff (higher elastic modulus) rods within the hydrogel rod to increase the composite stiffness of the hydrogel rod. As above, non-swelling or minimally swelling second, third, or additional layers are applied to each composite hydrogel rod. Upon removal of the second, third, or additional layers, the hydrogel cores will expand. The composite first stressed layers will have enhanced mechanical strength with which to oppose compression of the adjacent tissue.

Third, the first stressed layers may be placed under compression or expansion by means of thermal expansion or contraction. For example, the first stressed layers may be placed under tension by first cooling it by thermal means including but not limited to refrigeration or freezing (e.g., by exposure to liquid nitrogen). While the first stressed layer remains cool, one or more stress-free second, third, or additional layers are applied. When the composite temperature is raised to ambient room or body temperature, the first stressed layer will ideally return to its stress-free state, while the second, third, or additional layers will have expanded considerably. Shear forces between the first stressed layer and the second, third, or additional layers will place the first stressed layer in tension and the second, third, or additional layers in contraction. Selective removal of the second, third, or additional layers will free the tension of the first stressed layer to act on the adjacent tissue, for example, by curving it.

Similarly, the first stressed layer may be placed under tension by first heating it by thermal means, including, but not limited to, placement in furnaces, near heat reservoirs, exposure to thermal radiation or warm convective fluid, etc. Heating below the melting temperature and/or the glass transition temperature may be preferential. Heating near the melting temperature and/or the glass transition temperature may be preferred. While the first stressed layer remains warm, one or more stress-free second, third, and/or additional layers are applied. When the composite temperature is lowered to ambient room or body temperature, the first stressed layer will ideally return to its stress-free state, while the second, third, or additional layers will have contracted considerably. Shear forces between the first stressed layer and the second, third, or additional layers will place the second, third, or additional layers in tension and the first stressed layer in contraction. Selective removal of the second, third, and/or additional layers will free the compression of the first stressed layer to act on adjacent tissue.

Similarly, the first stressed layer may be placed under tension or compression by first heating it by thermal means, including but not limited to placement in furnaces, near heat reservoirs, exposure to thermal radiation or warm convective fluid, etc. Heating near or above the glass transition temperature but not dramatically above the melting temperature will allow the first stressed layer(s) to thermally relax. While warm, a stress-free second, third, and/or additional layers are applied. When the composite temperature is lowered to ambient room or body temperature, the tension or compression of the first stressed layer relative to the second, third, or additional layers will depend on the coefficients of thermal expansion of the materials. If the second, third, and/or additional layers possess a coefficient of thermal expansion greater than that of the first stressed layer, then the first stressed layer will be placed under compression. If the second, third, and/or additional layers possess a coefficient of thermal expansion lower than that of the first stressed layer, then the first stressed layer will be placed under tension. In either case, selective removal of the second, third, or additional layers will free the compression of the first stressed layer to act on adjacent tissue.

Similarly, the first stressed layer may be placed under tension or compression by first cooling it by thermal means including but not limited to refrigeration or freezing (e.g., by exposure to liquid nitrogen). Temperatures above the glass transition temperature of the first stressed layer are preferred to allow this layer to thermally relax. While the first stressed layer is still cool, a stress-free second, third, and/or additional layers is/are applied. When the composite temperature is raised to ambient room or body temperature, the tension or compression of the first stressed layer relative to the second, third, and/or additional layers will depend on the coefficients of thermal expansion of these materials. If the second, third, and/or additional layers possess a coefficient of thermal expansion greater than that of the first stressed layer, then the first stressed layer will be placed under tension. If the second, third, and/or additional layers possess a coefficient of thermal expansion lower than that of the first stressed layer, then the first stressed layer will be placed under compression. In either case, selective removal of the second, third, and/or additional layers will free the tension or compression of the first stressed layer to act on adjacent tissue.

In these examples, greater differences in the coefficients of thermal expansion between the first stressed layer and second, third and/or additional layers are preferential. Polymeric materials are preferential for these applications because they often have relatively large coefficients of thermal expansions relative to other classes of materials, though other materials remain feasible and within the scope of the present disclosure.

Fourth, the first stressed layer may be placed under compression by beginning with a hollow elastomeric first stressed layer upon which a second, third, and/or additional layer is/are fixed. One end of the first stressed layer is capped while the other is attached to a pressure producing device including but not limited to a pressurized air cylinder, air pump, compressor, liquid pump, etc. Fluid enters the first stressed layer and hydrostatic pressure leads to at least partial expansion, restrained at least partially by the second, third, and/or additional layers. The pressure end of the first stressed layer is then cauterized or cleaved without loss of seal and then more completely sealed, if necessary. In this manner, the first stressed layer is placed under compression whereas the second, third, or additional layers are under tension. Selective removal of the second, third, and/or additional layers frees the compression of the first stressed layer to act on adjacent tissue.

Fifth, the contracting compositions may be placed on a rigid minimally to negligibly contracting or minimally to negligibly expanding frame (see FIG. 15). The frame comprises interdigitating elements 1502 and 1504 (with adjacent frame elements) that are connected by compositions 1506 of a first length. As the second, third, and/or additional layers degrade or remove, the fiber length decreases pushing the interdigitated elements apart to expand the net dimensions of the composite structure (see FIG. 15). If the compositions vary along their lengths then curvature develops. These structures may be preferentially used to make gradually expanding stents.

Sixth, in various embodiments, the layers may be preferentially fabricated by extrusion with or without movable dyes, microfluidics, deposition, stamping, lithography, embossing, hot melt, cold melt, wet spinning, printing, melting sequential layers, layer-by-layer deposition/dip coating methods, evaporative deposition, selective oxidation of external surfaces, inter alia. In some embodiments, the first stressed layer may be fabricated by extrusion with or without movable dyes, microfluidics, deposition, stamping, lithography, embossing, et cetera. In some embodiments, the second, third, and additional layers may be applied by hot melt, cold melt, wet spinning, printing, melting sequential layers, layer-by-layer deposition/dip coating, evaporative deposition, oxidation of the core (e.g., for PDMS), glued with cyanoacrylates, polymerization on surface at room temperature, enzymatic polymerization, inter alia. In some embodiments, one or more layers are prepared in a mold. In some embodiments, two or more layers are woven (e.g., Irish knots, auxetic knots, etc.), knitted, threaded, printed, sculpted, molded, stamped lithographed, glued (e.g., cyanoacrylates), deposited, annealed, fried, or otherwise formed into the initial composition. In various embodiments, the layers are comprised of sheets that are punctured or stamped with or without forms. In various embodiments, the layers are annealed together.

In various embodiments, the layers are printed. In some embodiments, a 3D printer prints each layer sequentially. In some embodiments, one or more layers are printed on strained or compressed substrates. For example, the substrate may comprise central fibers, dried compressed foams, stretched latex, stretched elastic sheets, etc. In some embodiments, the one or more second layers are printed on one side of a first stretched layer. In some embodiments, one or more third layers are printed on another side of the first stretched layer. In some embodiments, the first stretched layer is flat. In some embodiments, the first stretch layer or compositions possess a curvature onto which the printer prints more layers.

Notably, if the first stressed layer is in tension and the second, third, and/or additional layers are in compression, degrading the first stressed layer first provides a way for expansion, while if the first stressed layer is in compression and the second, third, and/or additional layers in tension, the composition will contract as the first stressed layer selectively erodes.

Combinations of the above formulations and preparation (i.e., thermal, swelling, and mechanical) are also feasible in all their varieties. For example, a first stressed layer may be clamped, stretched, and cooled prior to application of the second, third, and/or additional layer(s), such that upon warming to room or body ambient temperature, the first stressed layer will be placed in tension. The combination allows enhanced tension not readily achievable without the combination. Similarly, a first stressed layer comprising a dry hydrogel may be heated and, while at temperature, be coated with a stress-free second, third, and/or additional layer(s). Upon cooling and exposure to solvent, the first stressed layer will be placed in compression. Alternatively, combinatoric formations and combinations not specifically enumerated herein lie within the scope of the present disclosure.

Some applications may call for multiple levels of timed tension or compression or combinations thereof. Multiple levels of tension can be achieved by placing the first stressed layer at a first level of tension or stretching to a first length. A second layer is applied. The first stressed and second layers are then stretched to a second level of tension or stretched to a second length, where the second length is greater than or less than the first length. A third layer is applied. Successive layers at successive tensions or length may be applied. In another embodiment, a continuous or small stepped gradient of subsequent layers may be applied at a continuous or small stepped gradient of lengths or tensions.

Similarly, a first dry hydrogel may comprise the first stressed layer of a composite. A second layer of a second dry hydrogel material may be applied to the first, wherein the swelling expansion in aqueous media of the first hydrogel is greater than that of the second hydrogel. Successive hydrogel layers may be applied in like manner. Finally, a non-swelling or minimally swelling coating or layer is applied. When hydrated, the first stressed layer will be under the greatest compression followed by the first internal layer, second internal layer, and so forth. Selective removal of each successive layer will act on adjacent tissue as discussed above in successive fashion.

Successive layers with greater or less compression or tension may be applied in varieties and combinations of the above methods and permutations and combinations thereof.

Compositions that apply both tension and compression at respective times also lie within the scope of the present disclosure. In at least one embodiment, a hydrogel first stressed layer is encased in a non-swelling or minimally swelling second layer. The composition is stretched to a preferred length or to a preferred tension. A third stress-free layer is applied. The tension is released such that the second layer is in tension while the third or outer layer is in compression. Selective removal of the outer layer releases the tension stored in the inner layers. Subsequent selective removal of the second layer with hydration of the hydrogel releases the compression stored in the first layer.

In at least one embodiment, the first stressed layer is placed under compression by thermal processing as discussed above and fixed with a second layer at a first preferred temperature. The composition is then placed in tension by thermal processing at a second preferred temperature. The tension is fixed in place by another stress-free layer. At ambient room or body temperature, the first stressed layer is under compression, while the first second layer is under tension. Selective removal of the outer layer releases tension to the tissue, while selective removal of the second layer releases the desired compression. Similar multiple layer constructs to achieve successive levels of tension or compression lie within the spirit and scope of the present disclosure.

Exemplary Applications

In various embodiments, the composition may be incorporated into sutures or suture materials. In at least one embodiment, individual strips or monofilament fibers comprise a suture. In at least one embodiment, the individual strips or monofilament fibers are connected to a needle. In another embodiment, an assembly or collection of strips or fibers woven or arranged into a polyfilament fiber comprise a suture. In at least one embodiment, the polyfilament suture is connected to a needle. In at least one embodiment, the threads that comprise the polyfilament suture comprise two or more types of strips or fibers that may differ in geometry of their material properties. In at least one embodiment, the fibers or strips in the polyfilament suture are selected to provide a sigmoidal or quasi-sigmoidal contraction profile. In some embodiments, the sutures are self-tightening sutures (e.g., a fiber that ties itself into knots) or self-loosening sutures depending on the stresses. The rate at which the sutures form curvatures, unique structures, and change positions depends on geometric, material, and mechanical factors.

In various embodiments, the composition or structures disclosed herein may serve as tissue scaffolds. In some embodiments, synthetic mesh with timed release and/or tuned curvature may be combined with native tissue or cells. In various embodiments, the tissue or cells may reside on the composition. In various embodiments, the tissue or cells may reside in the composition. In various embodiments, the tissue or cells may reside at the composition's surfaces. In various embodiments, the scaffold dimensions and/or curvature change with time. In some embodiments, such compositions and combinations may be used for tissue scaffolding for patients or to correct developmental birth defects. In some embodiments, the dimensions of the scaffold increase as a pediatric patient grows.

In various embodiments, the compositions and structures disclosed herein can tune delivery of incorporated or enclosed molecules. In some embodiments, the fully 3D structures bend in time and change curvature. In some embodiments, multiple alternating layers 1602 can cause a cavity 1604 containing a pharmaceutical or biopharmaceutical agent to open and close periodically for several days much like a flower, opening and shutting according to circadian rhythms. In some embodiments, each layer may contain one or more pharmaceutical or biopharmaceutical agents such that, as the layer dissolves, the agent is released at its appropriate time(s). Each of these embodiments may be used for example to design both loading doses and maintenance doses and even dose escalation into the same structure (see FIG. 16).

In various embodiments, the compositions and structures described herein may be used as bandages. In some embodiments, the bandages are longitudinal (see FIG. 17a). In some embodiments, the bandages are circular (see FIG. 17b). In some embodiments, the bandages possess an irregular shape. In some embodiments, the bandages conform to the patient as presented. In some embodiments, the same bandage conforms to the patient after one or more contours of the patient's body change. For example, rural and battlefield medicine often requires bandages that can be shipped flat but must accommodate the curvature of the human body. Examples include but are by no means limited to incisions between the toes where curvature requires a saddle topology, the ear where there are multiple directions of curvature, and sealing limb stumps with cup-like shaped bandages following a battle field injury (see FIG. 17d). Each can be shipped flat but develop curvature 1710 as additional dissolvable layers 1708 are removed. Each of these bandages requires multiple levels of curvature that can be achieved in one or multiple layer sheets or meshes, wherein each layer comprises, for example, a different curvature.

In various embodiments, these compositions and structures may be used to bandage non-swelling injuries. In these cases, rapid application of the bandage and fixing the curvature is necessary with preferred times comprising less than a minute. In some embodiments, the bandage comprises at least two biodegradable polymers layers oriented orthogonally to each other (see FIG. 17d). In some embodiments, a first solvent solubilizes a first layer or composite layer without affecting a second such that the first layer's curvature can be induced and quenched to tune the amount of curvature without affecting the curvature of the second layer. In this manner both directions can be tailored independently for a patient simply by adding and removing the solvent. Solvents with sterile, antiseptic properties remain preferential.

In some embodiments, these compositions and structures may be used to bandage or for bandages for swelling surgeries and injuries (see FIG. 17). Here the timing of contraction is more gradual because edema associated with these injuries typically develops over 6 to 36 hours. Bandages that gradually contract across surface wounds due to blast or burn injury are needed. In particularly severe cases, conventional bandages either have to be removed, perhaps reinjuring and dislodging freshly-adhered cells critical for recovery, or sequentially tightened to control edema. In some embodiments, the bandage does not have to be removed. In some embodiments, the mesh or bandage allows for a swollen inflammatory phase but then gradually and controllably contracts across the site of injury to improved patient outcomes by minimizing interaction with the wound site to decrease nursing monitoring load. In some embodiments, the surface of the bandage is marked with clotting factors to staunch blood flow.

In various embodiments these bandages accommodate a patient's curve surfaces and/or interfaces. In specific embodiments, a damaged or injured appendage is scanned in 3D using state-of-the-art scanning equipment. In some embodiments, a 3D printer prints a bandage that form fits the injury and the local curvature of the patient. In some embodiments, the bandage is tailor made to each patient. In some embodiments, the patient and/or surgeon first choose a desired shape or structure for the resulting tissue. The printer then prints the corresponding bandage or implant. The surgeon places the bandage or inserts the implant. The bandage or implant develops the first desired curvature before during or after implantation. In some embodiments, the bandage or implant develops a second desired curvature at a subsequent time to the first desired curvature such that the tissue adopts the desired curvature. The printing process is particularly advantageous because it allows the mesh to be individualized for each patient and each surgery.

In at least one embodiment, the bandage or mesh may be comprised of two or more distinct types of compositions having different release times to precisely tune the overall degradation rate of the bandage or mesh. This is a biomimetic feature of the present disclosure For example, an in vivo extracellular matrix dynamically rearranges in response to internal and external stimuli. More specifically, in wound healing following an inflammatory phase, fibroblasts and/or myofibroblasts infiltrate the wound 1 to 4 days following initial injury, deposit type III collagen, and shrink the wound perimeter. Contraction proceeds at experimentally determined rates of up to 0.75 mm/day, typically peaks at 2 weeks, and can continue, albeit gradually, for months (Olsen, et al., Journal of Theoretical Biology 177 (1995) 113). Models of the interaction between fibroblast and myofibroblast in-migration and wound contraction find both theoretically and experimentally that contraction profiles are, at least partially, sigmoidal. Wound contraction may expedite the healing process by decreasing the amount of granulation tissue and extracellular matrix formation required in the healing of the wide wound by secondary intention, a very slow process. Despite the importance of wound contraction to patient healing, synthetic bandages, sutures and surgical implants do not incorporate this important feature. The present disclosure enables design of active surgical mesh that dynamically and controllably contracts, expands, or curves to reshape its local environment.

In at least one embodiment, the arrangement, populations, and characteristics of the pretensioned compositions within the mesh are comprised in such a manner as to achieve a sigmoidal contraction profile. In at least one embodiment, this may be achieved by including smaller compositions that erode or degrade quickly with larger and thicker ones eroding slower and more gradually. Alternatively, fibers of the same net diameter but varying outer layer thicknesses can be arranged so that a few have thin outer layers, most have intermediate outer layer thicknesses, and a few have relatively thick outer layer thicknesses so as to achieve a sigmoidal contraction profile. Indeed, a wide variety of compositions remain available to achieve sigmoidal, linear, or other contraction profiles.

In various embodiments, the present disclosure comprises a surgical system or a portion of a surgical system. This system overcomes the challenges in translating open surgery into minimally invasive procedures. These solutions are based on the following principles: (1) constructing the mesh using specific patterns of various time-released stressed compositions, the chronologic and spatial configurations and curvatures of the surgical mesh or implement can be designed and tailored to meet the structural and functional requirements for various body systems including diseased or injured bodily systems. (2) The surgical mesh 1802 or implements should mimic the material properties of the native tissue in the region to be repaired. For example, surgical mesh may comprise at least one collagen-like fiber or element and at least one elastin like fiber or element. In this regard, the tissue properties of fascia and other connective tissue, may be mimicked by leaving the collagen-like fiber(s) or element(s) limp until a critical stress is achieved whereat the fiber(s) or element(s) become taught. For example, various embodiments leave the stiffer fibers or elements in zig-zag conformations. Here we further disclose arranging stiffer collagen-like fibers or elements with hairpin turns 1802 or loops 1808 (see FIG. 18) such that the portion of the fiber or element not in the hairpin turn holds the initially desired stresses. In some embodiments, the portion of the collagen-like fiber or elements not incorporated in the loops or hairpin turns tunes the temporal evolution of the curvature, position, or conformation of the mesh as described above. In some embodiments, the binding 1806 that holds the hairpin together or links the sides of hairpin turns dissolves, degrades, or releases in any manner described above or known to those skilled in the art such that the hairpin turn opens up so that the collagen-like fibers or elements may relax when their initial tensioning is no longer required. In this manner the mesh is initially taut and holds the tissue exactly where the surgeon indicates, or is initially loose but then becomes taut so as to position, contour, and shape adjacent tissue. The collagen-like fibers or elements relax over time so that the elastin like fibers and native tissue begin to sustain organ weight as the collagen-like fibers relax. In some embodiments, the loops or hairpin turns open up at rates similar to changing mesh curvature. In some embodiments, the loops or hairpin turns open up at rates slower than the changing mesh curvature. The use of bio-mimetic mesh at least partially avoids or minimizes the risk of mesh erosion, contraction, and the associated complications of pain and infection. (3) The surgical system anchors to at least a portion of the tissue. In some embodiments, the surgical system anchors with sutures, glues, et cetera. In some embodiments, the system anchors with microadhesives, e.g., as disclosed by Lau, et al., in U.S. Provisional Patent Application No. 61/701,439, filed Sep. 14, 2012. Using these adhesives incorporated or adhered to the mesh (e.g., by suturing, tape, glues, etc.), the surgical mesh can be inserted into a small space without the need of extensive dissection to create a larger space for adequate visualization and to accomplish the maneuver of suturing or stapling without damaging underlying or surrounding tissue.

A specific example of the use of the disclosed system is for closure of a swollen open wound. Closing the wound with traditional mesh remains a challenging and care intensive process in which the wound is covered, the mesh tightened, the first mesh/bandage is removed, a second mesh/bandage is applied to the wound, the mesh is tightened, the second mesh/bandage is removed, a third mesh/bandage is applied, and so forth until the wound is closed and the swelling is reduced. Each time the mesh/bandage is removed, it carries with it the beginnings of wound healing as cells begin to naturally close off the wound. Each removal effectively reopens the wound, prolonging the healing process compared to alternative systems disclosed herein that do not require removal.

In some embodiments, the surgeon cleans the wound as much as possible, feasible, and/or reasonable. The surgeon then administers an antibiotic to attenuate, minimize and/or prevent infection. The surgeon then places the disclosed surgical system onto or into the wound (see FIG. 17). The system confers the following advantages. First, suturing traditional mesh or bandages onto swelling injuries is difficult because finding clean tissue is challenging and the edema weakens any anchoring. Dermal glues are similarly challenging to administer because of the lack of clean surfaces on the most injured tissues. Removing mesh or bandages attached with dermal glue requires removing one or more layers of tissue. The microadhesives 1702 indicated above are advantageous because they adhere to a variety of tissue surfaces whether regular or irregular. Removing the mesh simply requires local addition of concentrated sugar solutions to effect at least partial release, minimizing local tissue damage.

Second, the surgical system comprises mesh, fiber mesh, sheets, and the like 1704 that gradually contract across the wound. The microadhesive anchorings lie on either side of the wound and the timed-release aspects of the regular and fiber mesh gradually reduce the distance between these anchorings. Because many bodily structures possess distinct curvatures, programming curvature into the mesh as discussed herein is distinctly advantageous. In some embodiments, the surgical system includes a membrane or partially permeable membrane 1706 to control moisture loss. Third, the mesh may have biomimetic and scaffolding properties to induce the right type of tissue to form locally. This is important because traditional mesh often induce fibrosis whereas biomimetic compositions may suppress fibrosis formation. Alternatively, the mesh may be designed out of biomimetic polymers that dissolve completely in 1-4 weeks such that it does not impede further plastic and cosmetic repairs associated with the injury.

Various embodiments of the present disclosure may individualize plastic and cosmetic surgery. In some embodiments, for example, a patient may generate or select an image or images of the way they would like to look following plastic or cosmetic surgery. In various embodiments, the surgeon generates or selects one or more images of the way they would prefer the patient to look at the end of the surgery. In various embodiments, these images are converted into composition-related parameters including spatial dimensions and pre-stresses individualized structures for a specific patient's curvature (desired or current). In various embodiments, 3D scans of the current appendage or body of the patient are used to design a personalized mesh. In some embodiments, 3D printers uniquely tailor the mesh for localized curvature of the patient. The printing process is particularly advantageous because it allows the mesh to be individualized for each patient and each surgery. The surgeon inserts the implant or bandage. In some embodiments, the implant develops a first desired curvature before, during, or after implantation. In some embodiments, the implant develops a second desired curvature at a subsequent time to the first desired curvature such that the tissue adopts the desired curvature.

A specific example of the use of the disclosed system is for repair of urinary incontinence or pelvic floor disorders. In this embodiment, the mesh or sling designed for these applications (see FIG. 19) is implanted within the body. Following implantation, the second, third, or additional layers 1904 erode by hydrolysis, enzymatic digestions, bioerosion, or other means, gradually releasing the tension or compression stored in the first stressed layer. Control over material selection and mesh geometry governs the timing, magnitude, placement of the tension or compression applied to the adjacent tissue, and the temporal evolution of the intended curvature. Even though tissue support comprising the mesh may initially seem loose and lacking tension at the time of the repair, the gradual contraction of the mesh over time allows the overlying vaginal mucosa and underlying attached pelvic fascia time to accommodate and remodel the new tissue support to reduct the prolapse. This approach of gradually integrating endopelvic fascial support allows for optimal healing and repair without the need to abruptly apply tension to, and potentially over contract, the endopelvic fascial support as is the case with the current state-of-the-art pelvic prolapse surgery using natural tissue or mesh augmentation. Over tensioning of the mesh, such may occur in vaginal prolapse repair, may cause flattening of the contour of the vaginal wall and leading to dyspareunia. Indeed, the three-dimensional programming of the mesh to convert to the predetermined contour over time gives desirable contour to vaginal repair, for example. By using the timed-release dual fiber biomimetic mesh 1906 strategy, increasing levels of support can be provided to millions of women including elderly women, while minimizing or eliminating the potential for tissue erosion. In some embodiments, the entire mesh is biodegradable so that longer-term erosion can also be avoided.

In at least one embodiment, the mesh or sling can be strategically positioned at the white line using microadhesive mesh elements 1902. For example, in the case of trans-vaginal paravaginal repair, after separating the vaginal mucosa from the underlying endopelvic fascia, using sharp and blunt dissection, the edge of a mesh with the micro-adhesive material 1902 can be pushed toward the white line with a thin blunt ribbon, within only a thin (millimeters) space created precisely to accommodate this maneuver. This should not create significant tissue trauma or excessive bleeding. Using the micro-adhesive 1902 to attach the mesh to the white line and the endopelvic fascia is a surface action, with no need for any penetration beyond the surface attachment. This eliminates all the risks associated with penetrating injuries of the underlying vital structures such as blood vessels, nerves, bowel or urinary tract. This would be applicable, in one example, for the attachment of the mesh to the sacro-spinous ligament, which has nerve and vascular bundles right behind it. If penetration by suturing, staple, hook trocar or anchor of those vital structures occurs during the sacro-spinous ligament, major bleeding, retro-peritoneal hematoma or nerve injury can be substantial complications. In contrast, using microadhesive mesh elements avoids these complications because less dissection is required, allowing typical surgeons to approach the white line with more confidence. Once the mesh edge is against the white line, the glycolic/sugar covering of the micro-adhesive would dissolved, and the micro-adhesive will firmly attach the mesh to the white line, a solid supporting bony structure. Very minimal operating space is required, with minimal tissue trauma. Furthermore, fewer surgical steps are required to successfully complete the surgery as straightforward dissection replaces multiple intricate twists.

In further embodiments, the mesh comes in distinct sizes and with distinct tensions. To assist the surgeon in selecting the appropriate mesh dimensions, some embodiments include a device that serves as a selection guide. In some embodiments, the guide flexibly inserts into the two white line incisions. In some embodiments, the guide displays markings corresponding to the mesh that would be most appropriate to accommodate the initial and final lengths or initial and final curvatures. In some embodiments, the guide displays markings corresponding to the mesh that would be most appropriate to accommodate the initial and extents of contraction that correspond to initial and final curvatures.

A specific example incorporating the surgical advances disclosed herein is the case of endoscopic abdominoplasty (see FIG. 20). Here tightening the fascia and the underlying rectus muscles 2002, which are typically unacceptably stretched out, remains challenging. For instance, applying acute tightening by suturing might tear the tissue, preventing it from holding postoperatively or even during surgery itself. This remains especially true when the patient coughs or bears down during bowel movements that push abdominal contents against the sutured muscle and fascia layers, each of which will cause failure of the surgery. By using the micro-adhesive mesh segments 2006, the larger composite mesh 2004 can cover a larger area of the abdominal fascia overlying the rectus muscle without having to suture, which minimizes or eliminates the risk of penetrating injury. The mesh can be positioned properly, through endoscopic means, before adhesion to fascia occurs after the dissolving a glycolic/sugar covering on the mesh. There is no tensioning during surgery, so the patient should be more comfortable postoperatively. The timed-release mesh 2004 then slowly contracts, drawing the underlying fascia and muscle with it, in a tension neutral way, and allowing the tissue time to repair and accommodate these gradual changes. This more effective way to repair the abdominal protrusions can avoid the problem of breaking down of the repair postoperatively.

A specific example using the disclosed system is mastopexy (FIG. 21). Ptosis of the breast results from gravity pulling the breast down while a person is in an erect position, thereby lengthening the fascial tissue above the main mass of the breast. Curvature in this case remains critical. Flattening of the upper portion of the breast by tensioning the mesh in a linear or planar fashion or with traditional mesh causes unfavorable aesthetic effects. In some embodiments, the surgeon works with the patient to determine the current contours of the breast and the desired post surgical contours of the breast. An individualized mesh is designed that selects the initial and desired final curvatures and the rate at which the mesh will gradually transition between these curvatures using a timed-release composition 2106. Combinations of reentrant and traditional designs of the mesh are preferential because reentrant designs readily provide the synclastic curvature required near the apex of the breast while more traditional mesh designs are more appropriate to the anticlastic curvature between the apex and anchoring at the clavicle bone 2102, for example. This combination specifically avoids the unfavorable flattening achieved by traditional surgical mesh. The mesh further incorporates the microadhesive elements 2104 or they are sutured directly to the remainder of the mesh preoperatively. The microadhesive mesh minimizes the amount of dissection required to successfully complete the surgery. In this manner, the complicated and invasive steps associated with open surgery for conventional breast lifts may be avoided, reducing or eliminating the chance of scarring and infection.

The disclosed methods also do not require a long recovery time associated with conventional breast lifts as fewer smaller incisions have to heal. The bio-mimetic nature of the mesh system 2108 is also preferential because will give the desirable tissue feel of the repaired breast also. The breast will move, stretch and contour more naturally as the patient moves through different positions (e.g., prone to standing). Indeed, each of these features is not only preferential to mastopexy but also for breast replacement for breast cancer survivors, inter alia.

Another example of the disclosed system is a stent. The purpose of a stent is to open and/or keep open a cylindrical surface. These systems begin with one curvature and end with another curvature, often employing springs, balloons, or other systems to change the curvature of a lumenal surface. In various embodiments, the stent comprises a timed-release curvature system that gradually opens up the lumen. In other embodiments, the stent comprises a microadhesive. For instance, the microadhesive may be attached at one point, one region, one longitudinal region, etc. A specific example where this may be helpful is in the treatment of achalasia in which the lower esophageal sphincter remains abnormally closed. Insertion of such a stent may hold the sphincter open so that bolus flow proceeds naturally. Similar embodiments may be used to open and keep open other openings, conduits, or organs.

A face lift is yet another example where the disclosed system can be used to accomplish contour remodeling. The objective of a face lift, a surgical procedure conventionally performed by either open or endoscopic techniques, is to tighten and to rebalance the subcutaneous musculoaponeurotic system (SMAS) in specific directions over different zones of the forehead, face and/or neck. With conventional procedures, imparting directionality to the SMAS is generally accomplished by cutting and suturing the tissue in strategic areas along specific directions. Tightening the skin by suturing enables a surgeon to remodel different zones of the forehead, face, and neck to reverse the sagging or loosening of facial tissue caused by gravity and the aging process, resulting in a more youthful appearance. Controlling curvature is a key feature of this application. In various embodiments, the curvature mimics that of native tissue including, for example, curvature around jowls.

In some embodiments, the patient and surgeon determine the current curvature and the desired curvature as described herein. An individualized mesh selects the initial and desired final curvatures and the rate at which the mesh will gradually transition between these curvatures. A mesh system 2202 is generated and implanted (FIG. 22). In some embodiments the mesh is initially loose so that the patient appears almost normal when the surgery is complete. Over a reasonable period (e.g., days to months), the mesh contracts, expands and gradually contours the skin from beneath. In some embodiments, the surgeon uses energy sources to change the initial contour of the mesh. In some embodiments, the patient returns for regular (e.g., weakly) visits in which the surgeon applies energy to gradually (possibly artistically) contract the mesh after initial insertion and initial healing. By using the microadhesive mesh, even smaller incisions are required shortening healing times. When the mesh also incorporates biomimetic aspects, the tissue moves naturally as the patient moves their head through different positions.

The foregoing examples of abdominoplasty, breast lifts, stents, and face lifts are specific embodiments of clinical applications that benefit from the non-invasive tissue shaping techniques disclosed herein. There are, however, many other applications that can be used to treat conditions where the present disclosure may be useful or have a therapeutic or cosmetic effect. Other applications include, for example, brow and neck lifts, arm lifts, buttock or thigh lift, calf contouring, genital plastic surgery, vaginal tightening, etc.

The present disclosure further provides for monitoring the deformation of the structures remotely. This includes but is not limited to including microbubbles in the mesh for UV spectroscopy or ultrasound detection, incorporating metallic particles or staples within the structure for magnetic resonance imaging (MRI) or fluoroscopy, attaching metallic objects to the structure for computed tomography (CT), including radioactive labels for single photon emission computed tomography (SPECT) or gamma camera imaging, etc.

Self-assembly of macroscopic structures may be useful in a broad arrange of fields with a multiplicity of applications. For example, it may be useful in any field of human endeavor where human manipulation is challenging or limited (e.g., hard to reach spaces or where sterilization requirements are intense such as medical surgeries, nuclear tests, space exploration, subsea exploration, inside electronics micro/nano fabrication facilities, inside BSL III or IV facilities, etc.).

From the foregoing, it will be appreciated that specific embodiments of the disclosure have been described herein for purposes of illustration, but that various modifications may be made without deviating from the spirit and scope of the disclosure. Aspects described in the context of particular embodiments may be combined or eliminated with other embodiments. Further, although advantages associated with certain embodiments have been described in the context of those embodiments, other embodiments may also exhibit such advantages, and not all embodiments need necessarily exhibit such advantages to fall within the scope of the disclosure. Accordingly, the scope of the disclosure is not limited except as by the appended claims.

1. A stressed timed-release multilayer composite, comprising a first stressed layer, and a second layer and third layer that hold the first layer under said stress,
wherein the second and third layers are configured to at least partially change to release at least a portion of the stress of the first layer in response to the second layer and/or the third layer being at least partially changed.

2. The composite of claim 1, wherein the second and third layers are dimensionally symmetric.

3. The composite of claim 1, wherein the second and third layers are compositionally symmetric.

4. The composite of claim 1, wherein the second and third layers are not symmetric.

5. The composite of claim 1, wherein an imbalance in stress between the first layer and the second and/or third layers causes at least a physical curvature of the composite when the second and/or third layers are at least partially changed.

6. The composite of claim 5, wherein the curvature changes as the second and/or third layers change.

7. The composite of claim 1, wherein the second and/or third layers have an elastic modulus exceeding that of the first layer.

8. The composite of claim 1, wherein the second and third layers at least partially change by removal from the composite.

9. The composite of claim 8, wherein the second and third layers are removed at an equivalent rate.

10. The composite of claim 8, wherein the third layer is removed faster or earlier than the second layer.

11. The composite of claim 8, wherein the second and third layers are removable by at least one of erosion, degradation, biodegradation, bioerosion, photooxidation, photodegradation, delamination, or mechanical erosion.

12. The composite of claim 1, wherein at least one of the layers comprises a polymer, a hydrogel, or polyelectrolyte hydrogel.

13. The composite of claim 1, wherein one or more of the first, second, and third layers comprises one or more lamina.

14. The composite of claim 1, wherein the second and/or third layers at least partially change in dimension by swelling or release of molecules to or imbibition of molecules from a surrounding environment.

15. The composite of claim 1, wherein the second and/or third layers at least partially change in elastic modulus by at least one of swelling, changes in porosity, or release of molecules to or imbibition of molecules from a surrounding environment.

16. The composite of claim 1, wherein the first layer is further configured to change by at least one of biodegradation, bioerosion, photooxidation, photodegradation, delamination, or mechanical erosion.

17. The composite of claim 1, wherein the third layer at least partially changes by removal though loss of adhesion.

18. A mesh comprising one or more elements formed of the composite of claim 1.

19. The mesh of claim 18, wherein the one or more elements are composites tuned for a timed release of the stress.

20. The mesh of claim 18, wherein the one or more elements are composites tuned for a timed formation of a physical curvature.

21. The mesh of claim 18, wherein the mesh comprises a first plurality of elements formed of the composite of claim 1 having a first orientation, direction, or curvature, and wherein the mesh comprises a second plurality of elements formed of the composite of claim 1 having a second orientation, direction, or curvature.

22. The mesh of claim 18, wherein the mesh is comprised of a plurality of elements formed of the composite of claim 1, wherein the elements are configured in multiple orientations or directions, and wherein the elements are tuned for different timed release of the stress.

23. A medical device, bandage, implant, tissue construct, or sling comprising the mesh of claim 18.

24. The mesh of claim 18, wherein the mesh is configured using a pattern of stressed timed-release layers such that a chronological and spatial pattern of the one or more elements forming the mesh meets a structural and functional requirement for plastic or reconstructive surgery in a body system.

25. A stressed timed-release bilayer composite, comprising a first stressed layer and a second layer that holds the first layer under said stress forming a first physical curvature of the composite, wherein one or both of the first and/or second layers are configured to at least partially change and thereby form a second physical curvature.

26. The composite of claim 25, where a change in dimension of the composite of one or both of the first and/or second layers is caused by selective swelling, partial degradation of an interpenetrating network, or release of a plasticizing or other small molecules.

27-50. (canceled)
